(19) United States
(12) Patent Application Publication
(10) Pub. No.: US 2026/0345829 A1
(43) Pub. Date: Nov. 5, 2026

(54) TYROSINASE GENE THERAPY FOR OCULOCUTANEOUS ALBINISM TYPE 1A

(71) Applicant: The United States of America, as represented by the Secretary, Department of Health and Human Ser, Bethesda, MD (US)

(72) Inventors: Brian P. Brooks, Kensington, MD (US); Aman George, Gaithersburg, MD (US)

(73) Assignee: The United States of America, as represented by the Secretary, Department of Health and Human Ser, Bethesda, MD (US)

(21) Appl. No.: 19/486,763

(22) PCT Filed: May 24, 2024
(86) PCT No.: PCT/US2024/031101

§ 371 Date: Nov. 21, 2025

Related U.S. Application Data
(60) Provisional application No. 63/468,748, filed on May 24, 2023.

(51) Int. Cl. A61K 48/00; A61K 38/44; A61P 27/02; C12N 15/86

(52) U.S. Cl. CPC A61K 48/0058; A61K 38/44; A61K 48/0075; A61P 27/02; C12N 15/86; C12Y 114/18001; C12N 2750/14143; C12N 2750/14171

(57) ABSTRACT

The invention provides a genetic vector for expressing a gene of interest in tissues of the eye of a human patient, the vector comprising the gene of interest operably linked to a promoter suitable for effecting the expression of the gene of interest within the eye tissue (such as the RPE) of the human patient. The vector can be, for example AAV (e.g., AAV5); the gene of interest can be, for example, the human Tyrosinase gene and the promoter can be, for example, the human BESTROPHIN (hVMD2) gene promoter. Compositions comprising such vectors and methods of administering them, and effecting expression of the gene of interest within tissues of the eye also are provided. For example, the method can be used therapeutically, such as to treat human patients suffering from oculocutaneous albinism type 1A (OCA1A) or other clinically-defined pigmentation related condition due to a loss-of-function mutation in the Tyrosinase gene.

### CROSS-REFERENCE TO RELATED APPLICATIONS

This patent application claims the benefit of U.S. Provisional Patent Application No. 63/468,748, filed May 24, 2023, which is incorporated by reference.

### STATEMENT REGARDING FEDERALLY SPONSORED RESEARCH OR DEVELOPMENT

This invention was made with Government support under project numbers EY000470 by the National Institutes of Health, National Eye Institute. The Government has certain rights in the invention.

### BACKGROUND OF THE INVENTION

Oculocutaneous albinism is a genetically heterogenous group of conditions that affect pigmentation and vision. Specific to the present invention, oculocutaneous albinism type 1A (OCA1A) is an autosomal-recessive condition caused due to mutations in the TYROSINASE (TYR) gene and characterized by reduced or absent pigmentation of the hair, skin, and eyes. OCA1A patients have increased risk for UV-induced skin cancers including melanoma, as well as varying degrees of visual impairment including photo-aversion, nystagmus, and reduced best-corrected visual acuity—all of which can negatively affect quality of life.

The TYR enzyme catalyzes the first and rate limiting step of the melanin biosynthesis pathway and loss of function mutations in TYR result in partial or complete loss of melanin synthesis. Melanin is synthesized in subcellular organelles called melanosomes that are present in melanocytes (skin and choroid of the eye) and the retinal pigment epithelium (RPE) of the eye. The RPE exists as a monolayer tissue of post-mitotic cells, adjacent to the light sensitive neural retina and is imperative for the proper function of photoreceptors. Human RPE is a prime target tissue for gene therapy studies given its involvement in many degenerative diseases of vision, including age-related macular degeneration in addition to OCA1A. Indeed, replacement of an RPE enzyme, RPE65, currently is the only FDA-approved gene therapy for ophthalmic disease. In OCA1A patients, the pigmentation of the RPE and choroid is reduced or completely absent, which can be observed clinically as iris trans-illumination on ophthalmologic exam.

The TYR enzyme is a rational target for gene therapy mediated disease rescue in pigmented cell types of the human body given its manageable size (i.e., ~58 kDa protein), easily defined phenotypic readout (i.e., melanin pigment), and that even comparatively small amounts of enzyme produced through gene transfer can potentially lead to a significant change in pigment product. However, this has been difficult to achieve due to technical hurdles due, in part, to vector specificity and delivery technology. To date, only a single mouse study has been reported, in which subretinal injection of AAV2 particles carrying the TYROSINASE gene under the control of CMV promoter induced pigmentation in the RPE and the choroid of mice (Gargiulo et al. (2009). Molecular Therapy: The Journal of the American Society of Gene Therapy, 17(8), 1347-1354. doi./10.1038/mt.2009.112). The present invention addresses these technical difficulties in therapeutic gene therapy to the human eye, especially the RPE.

### BRIEF SUMMARY OF THE INVENTION

In one embodiment, the invention provides a genetic vector, such as an adeno-associated virus (AAV) vector, for expressing a gene of interest in tissues of the eye of a human patient, the vector comprising the gene of interest operably linked to a promoter suitable for effecting the expression of the gene of interest within the eye tissue (such as the RPE) of the human patient. Compositions comprising such vectors and methods of administering them, and effecting expression of the gene of interest within tissues of the eye also are provided.

The method can be employed therapeutically, such as, for example, in embodiments wherein the gene of interest encodes the human TYR enzyme and the patient suffers from OCA1A or other clinically-defined pigmentation related condition due to a loss-of-function mutation in the gene encoding the TYR enzyme, wherein the inventive method can be used to induce pigmentation in the eye tissue of the patient. The nucleic acid encoding the human TYR enzyme can be administered using a genetic vector, such as an AAV vector or other vector platform. Furthermore, in the context of the invention, preferably the human TYROSINASE gene is under the control of (e.g., operably linked to) the human BESTROPHIN (hVMD2) gene promoter. Moreover, while the mode of delivery to the patient can be as selected by a competent physician, a preferred route for delivery of the vector is via suprachoroidal injection.

### BRIEF DESCRIPTION OF THE SEVERAL VIEWS OF THE DRAWING(S)

FIG. 1. Effect of AAV particle dose on transduction efficiency, RPE cell viability and GFP expression. (Panel A) Live RPE monolayer tissue transduced with AAV expressing GFP under the control of VMD2 promoter. (Panel B) Percentage of GFP+ cells and live cells two weeks post transduction of AAV particles carrying the hVMD2-GFP. (Panel C) Percentage of GFP+ cells and live cells four weeks post transduction of AAV particles carrying the hVMD2-GFP. (Panel D) Retrospective comparison of percentage of GFP+ cells and mean GFP fluorescence at two and weeks post transduction of AAV particles carrying the hVMD2-GFP.

FIG. 2. Effect of AAV particle dose on RPE marker gene expression and monolayer tissue integrity. (Panel A) Confocal images of fixed RPE monolayer tissue treated with increasing dose of AAV titer for 4 weeks and immuno-stained with Tight junctions marker-ZO1, Adherens junction marker-β-Catenin, melanosome marker-TYRP2 and RPE cell specific visual cycle enzyme-RPE65. transduced with AAV expressing GFP under the control of VMD2 promoter. (Panel B) Fluorescence measurement strategy and graph for ZO1 relative fluorescent intensity (membrane/cytoplasm) with increasing AAV titer after 4 weeks of transduction. (Panel C) Effect of increasing AAV titer after 4 weeks of transduction on trans-epithelial resistance of live RPE monolayer tissue.

FIG. 3. Suprachoroidal injection of AAV particles in rat eye leads to RPE specific GFP expression by human VMD2 promoter. (Panel A) Suprachoroidal injection strategy in rat eye (* indicates optic nerve head). (Panel B) Rat eye fundus imaging showing GFP expressing in the posterior segment of the eye, four months post single suprachoroidal injection of AAV (~10¹⁰ vector genomes). (Panel C) Pigmented rat eye section immuno-stained with RPE65 antibody (left panel), and albino rat eye section immuno-stained with RPE65 antibody (middle panel) and Rhodopsin (RHO) antibody (right panel). (Panel D) AAV injected rat eye exhibiting GFP co-localization with RPE65 antibody staining and basal localization pattern to RHO antibody staining.

FIG. 4. Human VMD2 promoter drives GFP expression specifically in RPE like cells of human retinal organoids. (Panel A) Brightfield microscopy images of RPE like cells and photoreceptors in the live human retinal organoids. Corresponding fluorescent images of live human retinal organoids transduced with AAV-hVMD2: GFP and AAV-CRX: GFP. (Panel B) Confocal microscopy images of GFP expression in live human retinal organoids transduced with AAV-hVMD2: GFP and AAV-CRX: GFP, at day 8 and 14 post AAV transduction. (Panel C) Immuno-stained cryosections of human retinal organoids transduced with AAV-CRX: GFP, 14 days post AAV transduction showing GFP colocalization with RHO immunostaining. (Panel D) Cryosections of human retinal organoids transduced with AAV-hVMD2: GFP, 14 days post AAV transduction showing GFP colocalization with pigmented RPE like cells.

FIG. 5. AAV mediated TYROSINASE delivery to OCA1A-iRPE monolayer tissue induces pigmentation. (Panel A) Brightfield microscopy of untreated and AAV-hVMD2: TYR transduced live OCA1A-iRPE monolayer tissue. (Panel B) Quantification of pigmented cell density in AAV-hVMD2: TYR transduced live OCA1A-iRPE monolayer tissue. (Panel C) Confocal microscopy of untreated and AAV-hVMD2: TYR transduced and fixed OCA1A-iRPE monolayer tissue. (Panel D) 2-dimensional tarns-well culture of untreated and AAV-hVMD2: TYR transduced OCA1A-iRPE monolayer tissue and iRPE derived from unaffected individuals.

FIG. 6. Transmission electron microscopy (TEM) of untreated and AAV-hVMD2: TYR transduced OCA1A-iRPE monolayer tissue. (Panel A) Representative TEM of untreated CTRL-iRPE monolayer tissue. (Panel B) TEM of untreated and AAV-hVMD2: TYR transduced OCA1A-iRPE monolayer tissue, showing presence of pigmented melanosomes in treated monolayer tissue. (Panel C) quantification of total melanosome (immature+mature melanosome) number in TEM images of untreated and AAV-hVMD2: TYR transduced OCA1A-iRPE monolayer tissue (N=3). (Panel D) Quantification of immature melanosomes in TEM images of untreated and AAV-hVMD2: TYR transduced OCA1A-iRPE monolayer tissue. (Panel E) Quantification of mature melanosomes in TEM images of untreated and AAV-hVMD2: TYR transduced OCA1A-iRPE monolayer tissue.

FIG. 7. Expression pattern of TYR, pMEL17 and TYRP2 in untreated and AAV-hVMD2: TYR transduced OCA1A-iRPE monolayer tissue. (Panel A) Confocal microscopy images of TYR immuno-staining and quantification of intensity and volume of fluorescent puncta (N=3). (Panel B) Confocal microscopy images of pMEL17 immuno-staining and quantification of intensity and volume of fluorescent puncta (N=3). (Panel C) Confocal microscopy images of TYRP2 immuno-staining and quantification of intensity and volume of fluorescent puncta (N=3).

FIG. 8. Suprachoroidal injection of AAV-hVMD: TYR in rat eye induces melanin pigment in retinal pigment epithelium and choroid. (Panel A) Fundus photograph of un-injected albino rat eye, with corresponding hematoxylin and eosin stained histology section showing no pigment in RPE (arrowhead). (Panel B) Fundus photograph of AAV-hVMD: TYR injected albino rat eye, with corresponding hematoxylin and eosin stained histology section showing pigment in RPE only (arrowhead). (Panel C) Fundus photograph of AAV-hVMD: TYR injected albino rat eye, with corresponding hematoxylin and eosin stained histology section showing pigment in choroid (arrowhead). (Panel D) Fontana Masson stained histology sections of un-injected pigmented rat (Long Evans) eye, AAV-hVMD: GFP injected albino rat eye and AAV-hVMD: TYR injected albino rat eye.

### DETAILED DESCRIPTION OF THE INVENTION

In one embodiment, the invention provides a vector, such as a lentiviral or an AAV vector, for expressing a gene of interest in tissues of the eye of a human patient, preferably within the RPE. The vector comprises the gene of interest operably linked to a promoter suitable for effecting the expression of the gene of interest within the eye tissue. The examples below employ an AAV vector, and while other vectors can be employed (such as lentiviral vectors), the invention will be described primarily with reference to AAV vectors.

An AAV vector according to the invention can be derived from any suitable AAV serotype (such as AAV1, AAV2, AAV3, AAV4, AAV5, AAV6, AAV7, AAV8, or AAV9, AAV10, AAV11, AAV12, AAV44.9, AAAV, BAAV, and others known in the art). However, a preferred vector for use in the present invention is AAV5.

The vector according to the present invention is engineered to employ a human promoter active within the RPE in operable linkage with a gene of interest. In this context, “operable linkage” is taken to mean that the promoter functions within the genome to promote the expression of the gene of interest. While any such promoter can be employed, a suitable promoter can comprise, for example, all or a portion of, or an active derivative of, a human BESTROPHIN gene promoter (hVMD2), or other promoters such as CAG and VMD2.

The gene of interest for use in the inventive vector can be any gene desired to be expressed exogenously within the human RPE tissue and able to be packaged, together with the promoter and any other genetic elements, into an AAV capsid (which has a capacity for carrying approximately 5 kB of DNA). Preferably (for example, to minimize immunological reaction against non-human proteins), the gene of interest encodes a human protein. The Examples presented below employ the human TYROSINASE (TYR) gene, the sequence of which is known (Accession: NM_000372.5), however, given the data presented in these Examples, it is envisioned that the inventive vector can suitably be employed to deliver other genes of interest as alternative genetic payloads (e.g., TYRP1-Accession: NM_000550.3, TYRP2-Accession: NM_001129889.3, and TH-Accession: NM_199292.3). Moreover, the gene of interest may vary from the exact native reported sequence of the gene, and its encoded protein can vary from reported sequences, so long as it retains the function of the native protein. Thus, the gene of interest may encode a protein that is at least 80%, such as at least 85%, such as at least 90%, such as at least 95%, such as at least 97%, such as at least 99% identical, or 100% identical to a native human protein (such as having such level of identity to the native human TYR gene or protein).

The inventive vector can be manufactured by methods known to those of ordinary skill. For example, an AAV vector comprising the human promoter (e.g., hVMD2)/gene-of-interest (e.g., human TYR gene) construct, flanked by AAV inverted terminal repeat elements (ITRs) can be co-transfected into a suitable packaging cell line (such as, but not limited to 293 cells) along with a second vector comprising the AAV rep and cap genes (for generating AAV capsids comprising the human promoter (e.g., hVMD2)/gene-of-interest (e.g., human TYR gene) construct and a suitable helper virus (typically an adenovirus or herpes simplex virus). Within such cells, the vectors replicate and produce AAV capsids containing the human promoter (e.g., hVMD2)/gene-of-interest (e.g., human TYR gene) construct within them. Thereafter, the cells can be lysed, and the capsids purified by standard methods (e.g., a CsCl gradient).

However, produced and purified, the inventive genetic vectors can be included in various desired compositions. Accordingly, the invention also provides a composition comprising the inventive genetic vector and a carrier (e.g., a pharmaceutically or physiologically acceptable carrier). For example, such compositions can include an aqueous solution, such as a physiologically compatible buffer. Examples of excipients to be included in the compositions include water, saline, Ringer's solution, and other aqueous physiologically balanced salt solutions. In some aspects, excipients are added to, for example, maintain particle stability or to prevent aggregation. Examples of such excipients include, but are not limited to, magnesium to maintain particle stability, pluronic acid to reduce sticking, mannitol to reduce aggregation, and the like, known to those skilled in the art.

Compositions are conveniently formulated in a form suitable for administration to a subject, if the inventive genetic vector is to be used in vivo. Techniques to formulate such compositions are known to those skilled in the art. For example, the inventive genetic vector can be combined with saline or other pharmaceutically acceptable solution. In some aspects, excipients are also added. In another aspect, a composition comprising inventive genetic vector is dried or lyophilized (and can contain a cytoprotectant, such as trehalose), and a saline solution or other pharmaceutically acceptable solution can be added to the composition prior to administration.

For administration to a subject (i.e., for in vivo use), the composition can, in addition to the inventive genetic vector, comprise other agents as desired, such as adjuvants, preservatives, and pharmaceutically active agents (e.g., drug substances or therapeutic biological agents). In such applications, the formulation can be selected to be suitable for the desired use. For example, the inventive vector and other desired components within the carrier can be formulated for topical, transdermal administration, or administration by injection, inhalation, or other known technique. In this context, a “subject” typically is a human subject (e.g., a patient), but the inventive vector can be delivered to a non-human animal, such as a companion animal (cat, dog, horse), a laboratory animal (e.g., a rat (as in the Examples below), a mouse, a non-human primate), or other animal. For use in a human subject, the inventive genetic vector can serve as a therapeutic agent to deliver desired proteins via gene-transfer applications involving the gene(s) encoding protein(s) of interest (“gene therapy”).

For in vivo use, especially for medical use, the dose of the composition comprising the inventive genetic vector to be administered to a subject to be effective will depend on the subject's condition, manner of administration, genetic payload (e.g., gene of interest and specific promoter, together with other genetic elements), and judgment of the prescribing physician.

Often a single dose can be sufficient; however, the dose can be repeated if desirable. In general, the dose can range from about 10⁴ AAV capsids per kilogram to about 10¹³ AAV capsids per kilogram. A preferred dose is in the range of from about 10⁶ AAV capsids per kilogram to about 10¹² AAV capsids per kilogram. A more preferred dose is in the range of from about 10⁸ AAV capsids per kilogram to about 10¹² AAV capsids per kilogram.

It will be understood by persons of ordinary skill in this field that the inventive genetic vector is primarily used to introduce and express transgenes within cells, either in vivo as noted above (e.g., for medical or veterinary uses, or for laboratory studies in animals), as well as in vitro. For any such use, in general, the inventive vector is administered to a cell, or a population of cells in vivo or in vitro, under conditions for the inventive vector to contact and infect the cell or population of cells. Thereafter, the genetic vector delivers its payload of genetic material (such as including AAV genomes comprising desired transgenes, for example) within the infected cell(s).

Once inside the cells, the transferred genome of the inventive genetic vector is able to be processed by the machinery of the infected cell(s) to express the gene of interest to produce the protein. Where the gene of interest is of therapeutic importance, the inventive method provides a method of treating a human patient by infecting cells within tissues of the patient's eye (such as the RPE) with the inventive vector. Within the infected cells, the gene of interest is expressed to deliver the therapeutically important protein to the cells within the impacted tissue. Thus, while by no means limiting, for example when the gene of interest encodes the human TYROSINASE protein, infecting tissues of the RPE with an inventive vector having such a gene, such as under the transcriptional control of (i.e., “operably linked to”) a promoter such as hVMD2, can effectively treat a patient suffering from a disease, such as OCA1A or other or other clinically-defined pigmentation related condition due to a loss-of-function mutation in the gene encoding the TYR enzyme.

While any suitable method can be employed to deliver the inventive vector to the RPE, in a preferred embodiment, the method for delivering the inventive vector to a patient employs the recently developed suprachoroidal injection technique. In performing this technique, if desired, multiple injections can advantageously be employed during a single surgical procedure to increase the amount of AAV particles being delivered and target multiple regions of the eye.

Several recent studies have reported on the use of suprachoroidal injection for various ocular diseases, especially in the setting of small molecules such as steroids. Most ocular gene therapies currently under investigation, however, are delivered via intravitreal or subretinal injection. Suprachoroidal injection offers several advantages along with some drawbacks when compared to these other methods of gene therapy delivery. While intravitreal injection provides a relatively non-invasive approach, viral transduction and immune response represent significant challenges. The presence of the internal limiting membrane limits the ability of injected viral vectors to transduce deeper layers of the retina including the photoreceptors and RPE. Additionally, viral injection into the vitreous cavity results in a significant humoral immune response when compared to subretinal injection. Subretinal injections are therefore currently favored as they allow for transduction of deeper retinal layers with minimal immune response. However, subretinal injections require a pars plana vitrectomy as well as the creation of a retinotomy and temporary retinal detachment, as a result are significantly invasive. This can be particularly problematic, as patients requiring retinal gene therapies already may have significant retinal damage.

Suprachoroidal injections overcome many of the issues with these other injection methods and are becoming a viable option for the treatment of numerous ocular diseases. For example, suprachoroidal triamcinolone is already being utilized to deliver steroids for the treatment of macular edema in humans with promising safety and efficacy data and is in trials for a variety of other retinal conditions. Animal studies have also shown promise for the use of these injections to deliver anti-VEGF molecules. Also, studies, including those presented below in the Examples, demonstrate that AAV particles injected in the suprachoroidal space can effectively transduce both the RPE and/or photoreceptors. Pertinent to this study, Ding et al. recently showed that AAV8 could transduce wide swathes of the RPE in rodents, pigs and nonhuman primates via suprachoroidal injection, raising the possibility of treating larger areas of the retina than previously obtained using subretinal injection, although there may be preferential distribution in the periphery compared to centrally.

Suprachoroidal injections also are advantageous in that they can be performed in-office with the use of topical anesthesia, without entering the vitreous cavity, and without disrupting the retina. Indeed, tools like the recently approved SCS microinjector³² (which can be employed in the context of the present invention) have helped make these injections straightforward, minimally invasive in-office procedures. This makes them significantly less invasive than the sub-retinal approach. The primary potential drawback of suprachoroidal injections could be their immunogenicity. The suprachoroidal injection of AAV8-CMV-GFP vectors into rhesus macaques resulted in an animal developing signs of anterior uveitis, peripheral chorioretinitis, and mild vitritis that resolved with oral corticosteroid treatment, but interestingly, the suprachoroidal injection of this vector led to a lower neutralizing antibody response than intravitreal delivery.^{30,31} However, the use of native (e.g., human) promotors and proteins, as in the context of the present invention, could lead to decreased immunogenicity, for example due to possible interspecific immunoreaction.

Furthermore, in albinism, reduced expression of proteins critical for melanin biosynthesis in the RPE and/or choroid results in failure of the fovea—the highly specialized area of the neural retina that gives humans high visual acuity—to fully develop.¹⁷ Although the reasons for this are unclear, one or more non-cell autonomous factors related to the process of melanin/melanosome formation results in abrogation of foveal maturation. Given that the foveal development continues postnatally,¹⁸ early delivery (e.g., wherein the human patient to be treated is a child yet undergoing foveal development) of a therapy, such as the inventive method, that increases pigmentation during the process of foveal development may result in improved visual outcomes. Additionally, the inventive reagents and methods can be adapted for delivery to melanocytes.

### EXAMPLES

The following experimental Examples further illustrate the invention but, of course, should not be construed as in any way limiting its scope.

The experiments reported in the Examples first demonstrate the utility of iPSC derived RPE, retinal organoids, and a rat model for determining the transduction efficiency, specificity, and toxicity of AAV5 particles. Next, the efficacy of AAV5-mediated delivery of TYROSINASE gene under the control of VMD2 gene promoter to induce pigmentation in OCA1A patient derived RPE monolayer tissue was confirmed. Subsequently, the efficacy of the in vivo efficacy, specificity, and toxicity of the AAV5 particles carrying the human TYROSINASE gene under the RPE-specific human VMD2 gene promoter was confirmed using suprachoroidal injection using a rat model.

The results of these experiments demonstrate an effective AAV-based strategy for selectively delivering target genes to human RPE monolayer tissue and, coupled with suprachoroidal injection technique, the platform can be used to effectively deliver genes, such as TYROSINASE gene, to the eye. These Examples provide the first proof of principal for rescuing pigmentation defects in human OCA1A patient-derived RPE cells, which faithfully recapitulate the patient phenotype. The data provide evidence using two different patient lines and one TYROSINASE gene knockout line, that the inventive strategy can be used for inducing melanin pigment without compromising RPE monolayer tissue morphology, junctional integrity, and viability.

The data generated in the experiments reported in the Examples below demonstrate that the RPE monolayer tissue can tolerate a dose of 10⁶ VG/RPE cells without significantly compromising junctional integrity of the monolayer and RPE marker protein, expression pattern. Using retinal organoids in vitro and rat eye in vivo, the inventive AAV delivery strategy resulted in RPE specific expression of GFP. Similar experiments led to pigment accumulation in both RPE and choroid or rat eye, when we delivered the TYROSINASE gene using AAV with the VMD2 promoter. Given that VMD2 is not reported to drive expression in choroidal melanocytes and that the same AAV serotype and plasmid backbone was used in these Examples for delivering both GFP and TYROSINASE, and without wishing to be bound by theory, these data are consistent with inter-cellular protein transfer of either TYROSINASE (but not GFP) mRNA/protein or melanosomes could potentially explain pigment accumulation in the choroid.

The data from these Examples also show that the inventive AAV strategy and construct can specifically transduce RPE but not the neural retina by exploiting human retinal organoids. As such, the present invention can be exploited not only to induce pigmentation but also target human RPE monolayer with other genes of interest. Given the involvement of RPE in many retinal degenerative disorders the inventive vector and method can be used to selectively target RPE in humans.

### Materials and Methods

The experimental work reported in the Examples employs, in part, an in vitro model of OCA1A in the form of RPE monolayer tissue, differentiated from patient-derived induced pluripotent stem cells (iRPE) (George et al., (2022) Stem Cell Reports, 17(1), 173-186. doi./10.1016/j.stemcr.2021.11.016). The in vitro iRPE model employed herein faithfully recapitulates these pigmentation defects that are observed in OCA1A patients. Multiple studies published in recent years signify the utility of iPSC derived RPE as an in vitro model to perform proof-of-principle studies for rescuing defects observed in the patient specific RPE.

The Examples below also involve the use of AAV5 vectors as a delivery system. Various AAV-based approaches, using other AAV serotypes, have been proposed for treatment of the eye, and some have been investigated using iPSC derived RPE. For example, one of the first studies 19 assayed multiple AAV serotypes (2/2, 2/4, 2/5, 2/8, and 2/9) for delivering the CHM gene to choroideremia patients'-derived iPSC-RPE. This study used a chicken β-actin promoter with a CMV (cytomegalovirus) enhancer (CAG) and observed that the AAV2/5 serotype had the highest transduction efficiency in human RPE. In 2017, Biogen sponsored a Phase 3 clinical trial assessing the delivery of an AAV2 vector into choroideremia patients. However, in 2021 it was reported that the clinical trial failed to see significant improvements in best corrected visual acuity. Brydon et al. tested the AAV2/2, AAV2/5, AAV2/Anc80 serotypes to deliver the pre-mRNA processing factor 31 (PRPF31) under a CMV promoter and a synthetic CASI promoter into iPSC-RPE derived from retinitis pigmentosa patients, and AAV2/Anc80 serotype under the CASI promoter displayed the highest transduction efficiency for restoring PRPF31 expression.²⁰ Another study used the AAV2 serotype to deliver the BEST1 gene under a CMV promoter into patient derived iPSC-RPE with BEST1 loss-of-function mutations, which causes a dominant macular dystrophy²¹. A similar study²² utilized the AAVS1 serotype to deliver the BEST1 gene under its own VMD2 promoter to iPSC-RPE derived from patients exhibiting Best vitelliform macular dystrophy otherwise known as BEST disease. For restoring RCBTB1 expression in RCBTB1-associated retinopathy,²³ AAV2 and AAV8 serotypes also have tested to deliver the gene using a CAG promoter and the woodchuck hepatitis virus post-transcriptional response element into patient iRPE. The authors observed that the AAV2 vector was more effective than the AAV8 vector for restoring RCBTB1 expression in RPE from patient carrying compound heterozygous RCBTBI mutations (c.170delG and C70delA). A recent study,²⁴ tested the AAV7m8 serotype to deliver the PRPF31 gene under a CAG promoter into human iPSC-RPE and retinal organoids harboring two PRPF31 mutations (Cys247X and Tyr90CysfsX21) causing retinitis pigmentosa.

In the performance of the experiments discussed below, the following particular materials and methods were employed.

Maintenance and Differentiation of Human iPSCs

All human iPSC related work was approved by NIH institutional review board protocol #11-E1-0245 (NCT01432847), and informed consent was obtained from all subjects. Fibroblast cells from clinically diagnosed and genetically confirmed (c.896G>A/c.896G>A and c.1118C>A/c.1467_1468insT) Oculocutaneous Albinism Type 1A OCA1A patients 7 were reprogrammed using Sendai virus-mediated delivery (#A16518, CYTOTUNE2, THERMOFISHER SCIENTIFIC, Carlsbad, CA, USA) of the four Yamanaka factors (c-MYC, KLF4, OCT4, and SOX2), following the manufacturer's recommendations. Routine culturing, expansion and, in vitro and in vivo characterization of iPSCs, has been described in detail earlier.⁷ Differentiation of iPSCs towards RPE using a developmentally guided protocol has been recently published.¹² Purified RPE cells were seeded onto trans-wells membranes (#3460, CORNING) for 6-8 weeks for maturation and formation of polarized monolayer tissue. All experiments were performed on these mature RPE monolayer tissues.

### Generation of Construct and AAV Particles

GFP or human TYROSINASE gene was sub-cloned into a pV5.2 plasmid downstream of hVMD2 gene promoter. Packaging of plasmid in AAV5 particles and purification has been described in detail previously¹³. AAV particles carrying the GFP or TYR constructs were added to the iRPE monolayer at different concentrations only once for 48 hrs. Post transduction, RPE monolayer tissue were maintained for multiple weeks, based on experimental design and media was changed every 48-72 hrs.

The trans-epithelial resistance (TER) measurements of iRPE monolayers were carried out using the STX2 electrode set with the EVOM2 meter (WORLD PRECISION INSTRUMENTS, Sarasota, Florida). Briefly, the electrodes (STX2, WPI) were applied on the apical side of iRPE monolayer and in the basal medium chamber, the current was passed and resistance values (Ohms) were noted down for each well. The unit area resistance (Ohms.cm²) is calculated by dividing TER to area of measurement (1.13 cm²). Student's t-test was performed to determine the level of significance.

Immunostaining of iRPE Monolayer Tissues

RPE tissue on membranes were washed three times with phosphate buffered saline (PBS) before fixing in 4% paraformaldehyde (PFA) for 15 minutes at room temperature (RT). Following three additional washes with phosphate buffered saline Tween 20 (PBST) (0.5% Tween 20 in PBS), cells were permeabilized with immunocytochemistry (ICC) blocking buffer (PBST, 0.5% bovine serum albumin [BSA], 0.05% sodium azide, 0.1% Triton X-100) for 1 hour. Cells were incubated with primary antibodies overnight at 4 °C. Samples were dark-incubated on a rocker for 1 hour. Following three PBST washes, samples were mounted on a glass slide with FLUOROMOUNT-G aqueous mounting medium (cat #0100-01; SOUTHERNBIOTECH, Birmingham, AL, USA).

### Transmission Electron Microscopy (TEM)

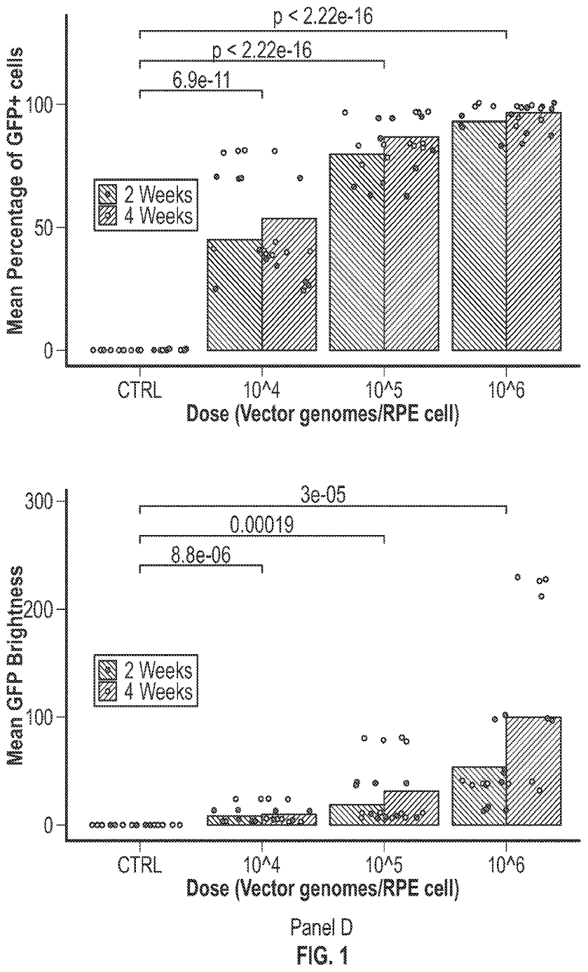

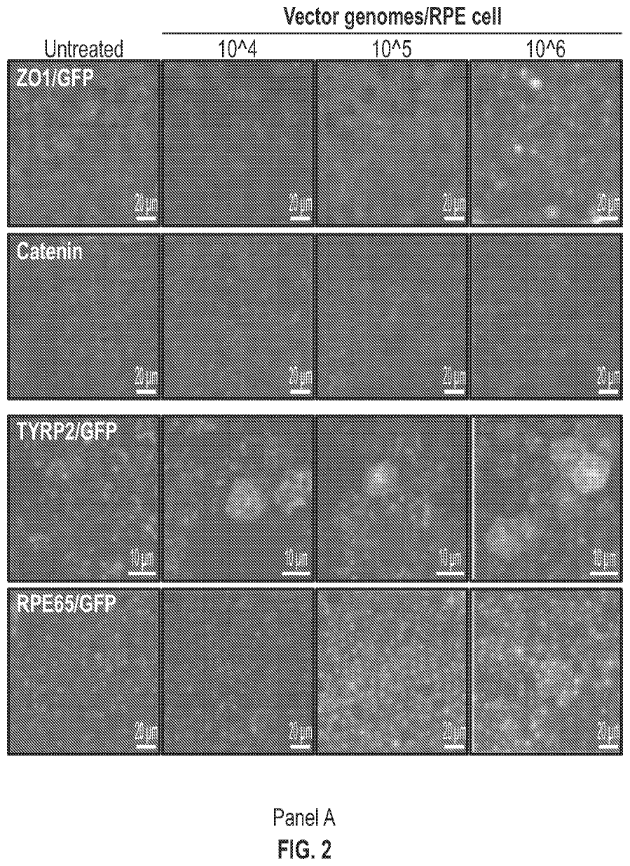

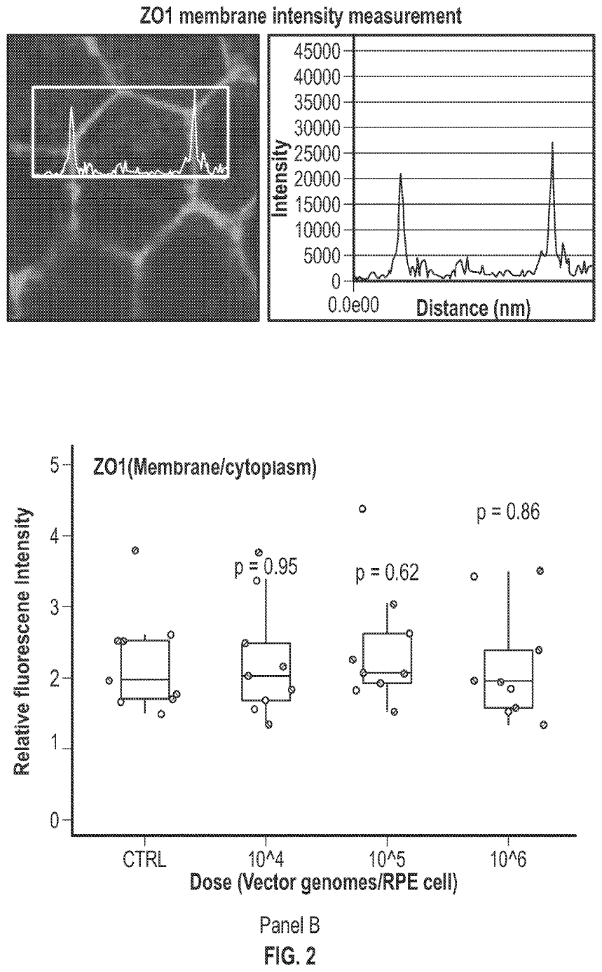

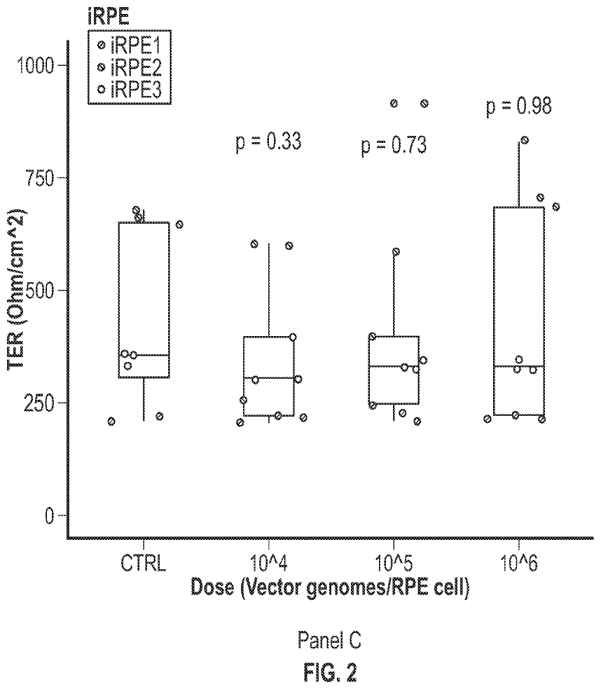

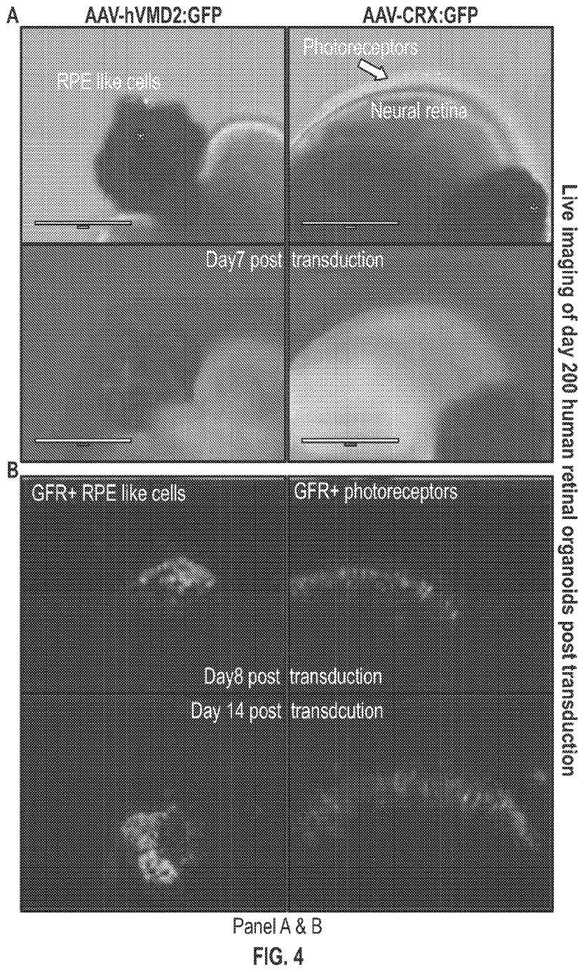

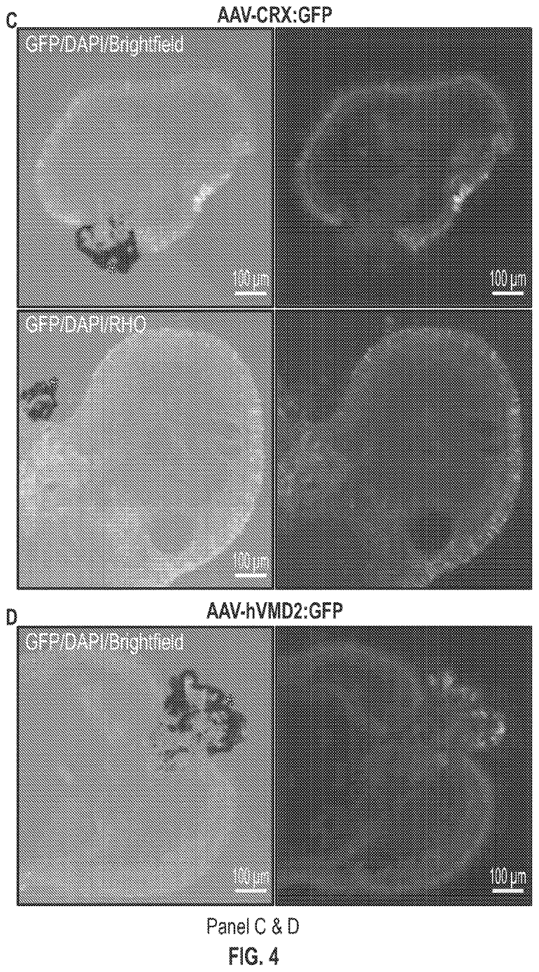

Confluent monolayers of iRPE cells grown on trans-wells were rinsed with PBS (3×, 5 min each), fixed in 2.5% buffered glutaraldehyde (ELECTRON MICROSCOPY SCIENCES, Hatfield, PA, USA) at room temperature for two hours, rinsed with PBS (3×, 5 min each), and post-fixed in 1% ice-cold buffered osmium tetroxide for one hour. Specimens were rinsed with PBS, dehydrated with an ethanol series (70%, 95%, and 100%, 5 min each) and propylene oxide (2×, 5 min each), and then embedded in epoxy resin. Thin sections, 90 nm thick, were collected on copper grids, air dried, doubly stained with uranyl acetate and lead citrate, and viewed with JEOL JM-1010 TEM. Images were acquired at 15k and 20k magnification to accommodate an RPE cell, and melanosomes observed in the image were counted manually. A single clone of four different control iPSC lines and a minimum of two different clones from each of the two OCA1A and the two OCA2 iPSC lines were differentiated towards RPE on trans-well membranes and then processed for TEM. At least five TEM photomicrographs of each monolayer were acquired randomly and used for quantification of different stages of melanosomes. The data was used to generate FIG. 2, Panel B, FIG. 2, Panel D and FIG. 4, Panel B, where each data point represents observations derived from a single TEM photomicrograph. Student's t-test was performed to determine the level of significance.

### Confocal and Brightfield Microscopy

For routine observation of iPSC and iRPE cell cultures and bright field imaging, ZEISS VERT.A1 (ZEISS, White Plains, NY, USA) inverted microscope was used. Confocal microscopy was performed using ZEISS LSM 880 and 700 microscopes.

### Flow Cytometry

Transduction efficiency was determined by measuring the expression of GFP after AAV-GFP transduction. RPE monolayers were detached from the TRANSWELLS plates in a two-step process: first wash away debris with 1xPBS three times and then incubated with this much TYRPLE for 30-45 mins. The RPE monolayer was disrupted and detached from the TRANSWELLS membrane by gentle pipetting. Cells were then collected in 1xPBS containing 2% FBS (FACS buffer) and centrifuged. This step was repeated 2 times. The cell suspension was filtered through a 50 um cell strainer. Cell viability was determined by exclusion of DAPI at a final concentration of 0.1 μg/ml. Data was acquired with a CYTOFLEX NUV instrument (BECKMAN COULTER, Brea CA) using the blue light excitation and 525 nm emission to detect GFP and violet light excitation and 450 nm emission to detect DAPI detection. Data analysis was done using CYTEXPERT software Version 2.5 (BECKMAN COULTER, Brea CA). Interesting cells were identified as DAPI negative, in the whole cell cluster in a FSC vs. SSC plot and being in a single cell state in the FSC-A vs. FSC-Width. Transduction efficiency was quantified as the Stain Index of GFP fluorescence intensity, which was calculated using the median fluorescent intensity and robust Standard Deviation as described.¹⁴

### Suprachoroidal Injection of AAV Particles in Rats

All animal studies were performed in accordance with the NEI/NIH animal ethics committee guidelines (NEI-696). Long Evans and Sprague Dawley (3-6 months old) were obtained from CHARLES RIVER. Rats were anesthetized using a mixture of ketamine and xylazine and secured to a surgical stage. For pupil dilation and reduction of eye movements, Tropicamide, Phenylephrine and Proparacaine (SANDOZ) were applied and eyes were kept lubricated using GenTeal® tears gel (ALCON). The eye was visualized under a ZEISS Stereo Dissecting Microscope. A custom device was wrapped around the base of the eye and secured with a vascular clip to induce proptosis. Once proptosis was induced, a small patch of conjunctiva 1 mm posterior to the limbus was removed, exposing the sclera. A 29-gauge insulin needle was used to create a partial-thickness pilot hole in the exposed sclera (four fifths of the way through the sclera). Then, a 33-gauge Hamilton needle (HAMILTON COMPANY) with a custom, flattened tip connected to a 10 μL Hamilton syringe containing AAV vector was introduced through the pilot hole, completely through the sclera, into the suprachoroidal space. With the needle in place, an assistant slowly pushed the syringe plunger and injected ~3-5 μL containing various dilutions of AAV5: VMD2-GFP or AAV5: VMD2-TYR into the suprachoroidal space. The needle was left in place for several seconds and then slowly withdrawn to minimize injection leakage. Antibiotic ointment NEO-POLYCIN (PERRIGO) and/or VETROPOLYCIN®HC (DECHRA) was applied to the eye post injection, and rats were injected with REVERTIDINE™ (MODERN VETERINARY THERAPEUTICS, LLC) to counter-act anesthesia.

Enucleated rat eyes were fixed in 1% formaldehyde and 1.25% glutaraldehyde. Histology was outsourced to HISTOSERV INC. (Germantown, MD), where eyes were embedded in paraffin, sectioned, and stained with Hematoxylin and eosin or Fontana-Masson stain. Eyes used for cryo-sectioning were treated in 10%, 20%, and 30% sucrose solutions before being embedded in cryomedia (TISSUE-TEK® O.C.T. Compound, SAKURA FINETEK) and frozen. 10-20 μm cryosections were placed on slides and immuno-stained (as mentioned above) before being imaged using confocal microscopy.

### Rat Fundus Imaging and Image Analysis

For fundus imaging rats were anesthetized as described above for long term sedation. Pupil dilation and stabilization of eye were performed as described above. The rat was then mounted on a stage, and a fundus imaging camera (MICRON II small animal in vivo retinal imaging system, PHOENIX RESEARCH LABORATORIES INC.) was used to take multiple brightfield and fluorescent images of eye where the injection occurred and near the optic nerve head to visualize GFP expression or pigment accumulation. From all the rat fundus images, the best quality image was chosen and used for analysis. ImageJ was used to calculate the GFP expression and pigmented patches by tracing the respective areas.

### EXAMPLE 1

This example demonstrates that AAV5 can efficiently and exclusively deliver a reporter gene (GFP) under the control of the human BESTROPHIN (hVMD2) gene promoter to human iRPE in vitro, rat RPE in vivo and to only RPE-like cells contained in human retinal organoids.

### AAV5 Efficiently Delivers GFP Driven by Human VMD2 Promoter to iRPE Monolayer Tissue

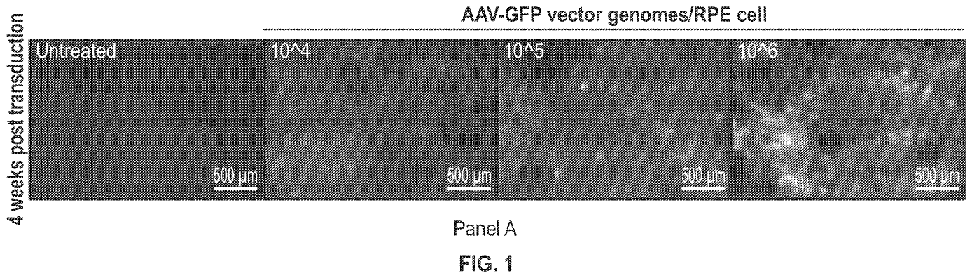

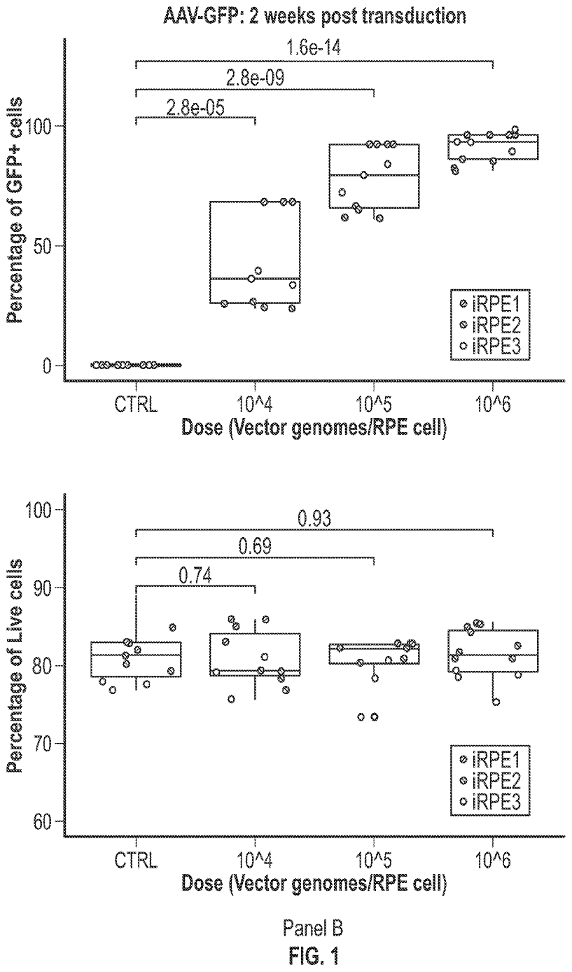

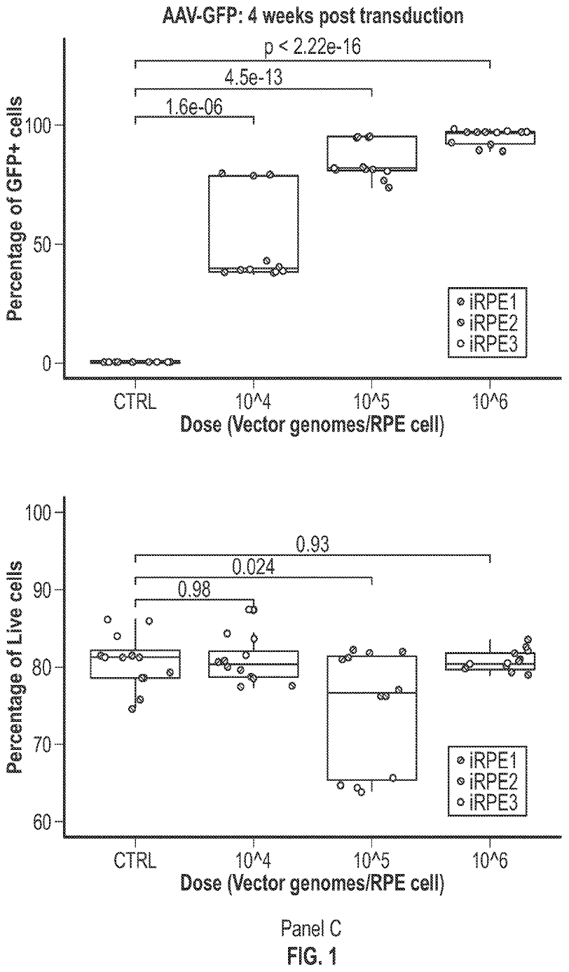

RPE cells were differentiated using a developmentally guided protocol as described previously. The differentiated RPE cells were seeded on to trans-well membranes and allowed to form a pigmented, polarized, and mature monolayer tissue, for 6-8 weeks. AAV5 particles carrying the GFP gene under the control of human BESTROPHIN gene promoter (hVMD2) were introduced on the apical side of the monolayer at different concentrations (10⁴, 10⁵ and 10⁶ vector genomes/RPE cell). 48 hrs post-AAV transduction cells were washed with 5% RPE media and cultured routinely for 4 weeks and imaged for GFP fluorescence every week (FIG. 1, Panel A). GFP expression can be observed as soon as 7 days post transduction, across all the three iRPE lines tested, GFP expression was never observed in untreated RPE monolayer. Two weeks post-transduction >70% cells of the RPE monolayer tissue were GFP+ at 10⁵ VG/cell and >90% cells were GFP+ at 106 VG/cell, as analyzed by flow-cytometry of live cells (FIG. 1, Panel B). We also observed an AAV dose dependent increase in the GFP fluorescence intensity of RPE cells, analyzed by flow-cytometry 2 weeks post transduction (FIG. 1, Panel D). We were able to achieve a transduction efficiency of >90% by 2 weeks post transduction with the highest tested dose of 10⁶ vg/cell without significantly compromising the viability of cells as measured via flow-cytometry, across the three iRPE lines tested (FIG. 1, Panel B). Fluorescence live cell imaging of RPE monolayer on trans-well membranes, 4 weeks post transduction exhibited a significant proportion of GFP+ cells (FIG. 1, Panel A). At the lowest dose of 10⁴ VG/cell<50% of RPE cells were GFP positive as measured by flow-cytometry, whereas at 10⁶ VG/cell more than 90% of RPE cells were GFP positive across the three iRPE lines tested (FIG. 1, Panel C). An AAV dose dependent increase in the GFP fluorescence intensity of RPE cells, as analyzed by flow-cytometry, was observed 4 weeks post transduction (FIG. 1, Panel D). The viability of total RPE cell population was not significantly affected due to the increasing AAV titer 4 weeks post transduction (FIG. 1, Panel C).

A comparison of transduction efficiency between 2 and 4 weeks suggested that similar efficiencies can be achieved by 10-fold lower dose (10⁵ vg/cells) at 4 weeks that is achieved by 10⁶ vg/cells titer in 2 weeks (FIG. 1, Panel D). This is particularly important as long term sustained expression from the plasmids might be required to rescue the disease phenotype and lower doses might fare better as compared to higher doses in terms of toxicity. Nevertheless, the fluorescence intensity was higher at 4 weeks compared to 2 weeks at all the concentrations tested (FIG. 1, Panel D). The forward and side scatter plots are reflective of the intracellular contents of the cells, due to the presence of significant number of melanosomes inside the RPE cell, the melanosomes tend to contribute most towards the side vs forward scatter profile, as compared to other intracellular organelles. We did not observe any significant shifts in the side vs forward scatter plots of the AAV treated iRPE cells, suggesting no significant changes in the melanosomal content of the RPE cells.

### RPE Monolayer Morphology and Integrity is not Compromised by AAV5 Transduction

To study the effect of increasing viral dose on RPE cell morphology, monolayer junctional integrity and single cell viability we performed immunofluorescence staining followed by confocal microscopy and trans-epithelial resistance (TER) measurements. As mentioned earlier, we did not observe a significant drop in RPE cell viability with an increasing viral dose at two-and four-weeks post transduction as studied by flowcytometry (FIG. 1, Panels B and C).

Live cell fluorescence imaging also did not reveal any observable changes in RPE monolayer tissues. To confirm this observation, we performed immuno-fluorescence staining of AAV treated RPE monolayers, 4 weeks post transduction, with a junctional protein marker ZO-1 and β-CATENIN, that labels apical tight junctions and Cadherin-junctions respectively. We did not observe any significant changes in the polygonal cell packing of RPE cells in the monolayer tissue as observed by ZO-1 and β-CATENIN localization pattern, using confocal microscopy (FIG. 2, Panel A).

We also performed ZO-1 immuno-staining fluorescence intensity measurements at the cell borders across all the three iRPE lines and observed no significant changes as compared to untreated iRPE monolayer tissues (FIG. 2, Panel B). No changes were observed in the staining patterns for mature melanosome marker TYRP2 and RPE65 proteins (FIG. 2, Panel A), as well. Next, we performed trans-epithelial resistance (TER) measurements, which is a measure of overall junctional integrity of RPE cells in the monolayer tissue. We did not observe any significant drop in the TER of iRPE monolayer tissues, with an increasing viral dose, suggesting that all AAV doses (10⁴-10⁶ vg/cell) were well-tolerated across all the three iRPE lines tested (FIG. 2, Panel C).

### Suprachoroidal Injection of AAV5 in Rat Eye Leads to RPE Specific Expression of GFP

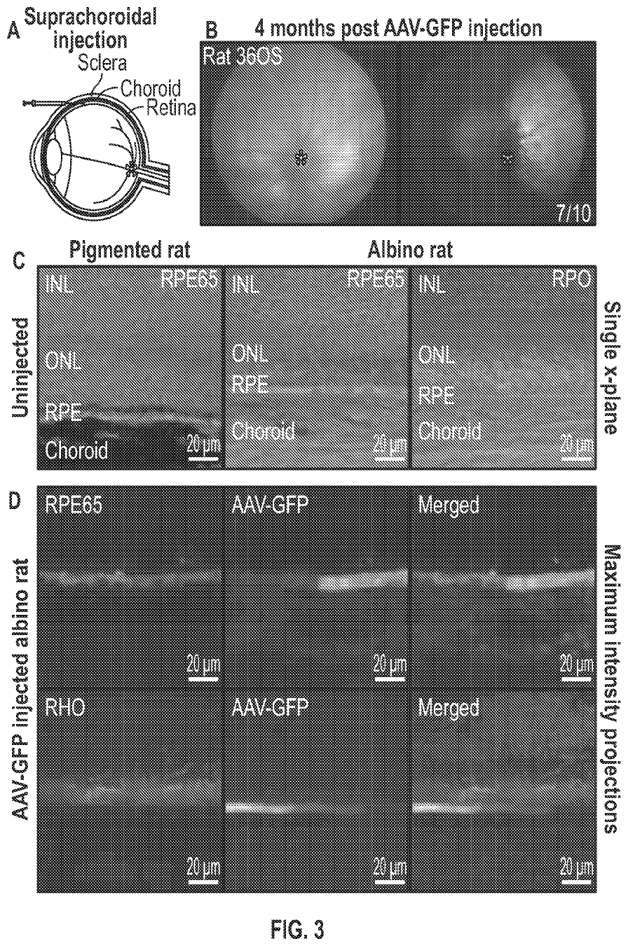

To study the in vivo localization specificity of GFP due to AAV5 transduction and GFP expression under the control of human VMD2 gene promoter we performed suprachoroidal injections (FIG. 3, Panel A) in pigmented Long Evans and albino Sprague Dawley rat eyes, that carry Tyrosinase gene mutation.¹⁵ We performed suprachoroidal injections of ~3 μL AAV5 particles carrying hVMD2-GFP construct (1.95×10⁸ VG/μL) in Sprague Dawley rat eyes. We observed GFP expression as soon as 2 weeks post injection (N=7/10 eyes) using fundoscopy (FIG. 3, Panel A). Four months post injection, GFP+ eyes (FIG. 3, Panel B) were dissected out and fixed for immuno-fluorescent staining, to study the specificity of GFP transfection. Un-injected rat eyes were used for optimizing antibody staining and RPE65 expression was observed only in RPE and (RHO) expression was localized to photoreceptor outer segments located on the apical side of the RPE (FIG. 3, Panel C). We then immuno-stained the AAV injected rat eyes with RPE65 and RHO and observed the GFP expression exclusively localized to the RPE monolayer in the rat eye (FIG. 3, Panel D, N=3 eyes), as observed via confocal microscopy. The boundary of GFP+ve and GFP−ve area of the injected eye is shown in FIG. 3, Panel D, to differentiate between immuno-staining and the AAV: hVMD2-GFP mediated GFP expression.

These observations suggested that the human VMD2 promoter can drive GFP expression exclusively in the rat RPE monolayer.

In the next set of experiments, we performed suprachoroidal injection of ~3 μL of sterile PBS only or of AAV5 particles carrying hVMD2-GFP construct (1.95×10¹⁰ VG/μL), to study injury caused due to the injection procedure and toxicity arising due to AAV introduction into the suprachoroidal space. Fundus imaging and histology of age matched rats was performed to account for the fixation and histology related artifacts. Fundus imaging for AAV injected eyes was performed 2 weeks post injection to observe GFP expression, prior to collection of eyes. GFP expression was observed in a localized area in most of the injected eyes (8/10 eyes, albino+pigmented), and there was significant variation in the level of fluorescence and area of GFP+ve patch. We performed extensive histological studies (~30 serial sections each eye) following paraffin-sectioning followed by Hematoxylin and eosin staining to study the damage arising due to injection procedure and toxicity of the AAV particles, compared to un-injected eyes. Two weeks post injection we observed scar tissue at the site of injection, in some cases as there was breaching of the choroid and RPE and loss of photoreceptors in the surrounding region, in both PBS only and AAV5 injected groups. Retinal detachment and blebbing of the neural retina were also observed in both groups. These defects were never observed in age matched un-injected rat eyes upon histological examination. Most of the defects arising due to suprachoroidal injections were shared among both PBS and AAV injected groups and we did not observe any additional defects in the AAV injected eyes. These observations suggested that, although non-traumatic suprachoroidal injections can be technically challenging, the AAV dose was well tolerated in the rat eye.

### AAV5 Transduction of Human Retinal Organoids Lead to VMD2 Driven GFP Expression in RPE

We next used human retinal organoids (~day 200) for testing the tissue specificity of AAV5 delivered hVMD2 driven GFP cassette, representing a human tissue with multiple cell types present. CRX promoter driven GFP cassette (CRX-GFP) packaged in AAV2 was used as positive control, which is known to transduce photoreceptors.¹⁶ We transduced the human retinal organoids (N=5) with AAV5 driven hVMD2-GFP and AAV2 driven CRX-GFP and maintained them for 14 days post transduction. We observed GFP fluorescence in both the groups as soon as 7 days post transduction (FIG. 4A). Confocal imaging of live transduced organoids exhibited RPE specific GFP expression in AAV5: hVMD2-GFP (5/5 organoids) treated organoids and photoreceptor specific expression of AAV2: CRX-GFP (5/5 organoids) in treated ones.

Two weeks post transduction, the organoids were fixed and cryosection and stained with RHO for visualization of photoreceptors via confocal microscopy. Retinal organoids transduced with AAV2: CRX-GFP expressed GFP only in the photoreceptors (N=3, FIG. 4, Panel C), which was further confirmed by immuno-staining of photoreceptors by RHO. Whereas the organoids treated with AAV5: hVMD2-GFP expressed GFP only in the pigmented RPE like cells (N=3, FIG. 4, Panel D. *) which was confirmed by presence of black pigmented clump of cells on the periphery of organoids. This observation suggests that hVMD2-GFP specifically express GFP in the RPE and not the neural retina. We also studied the photoreceptors in AAV2: CRX-GFP and AAV5: hVMD2-GFP treated organoids by staining with RHO and did not observe any significant changes in the expression pattern of RHO positive photoreceptors in the two treated groups (data not shown).

### EXAMPLE 2

This example demonstrates the efficacy of AAV5-mediated delivery of TYROSINASE gene under the control of VMD2 gene promoter to induce pigmentation in OCA1A patient derived RPE monolayer tissue.

### VMD2 Promoter Driven Tyrosinase Expression Induces Pigmentation in OCA1A Patient Derived iRPE

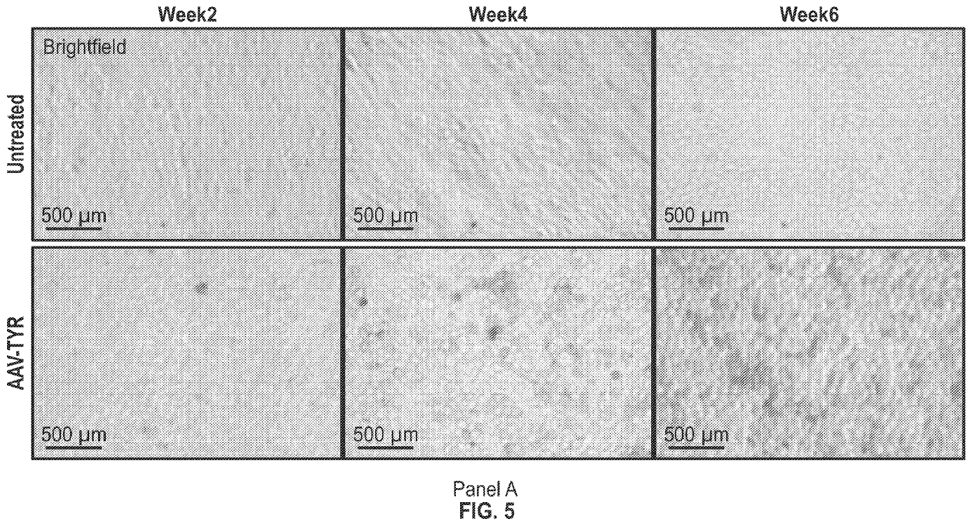

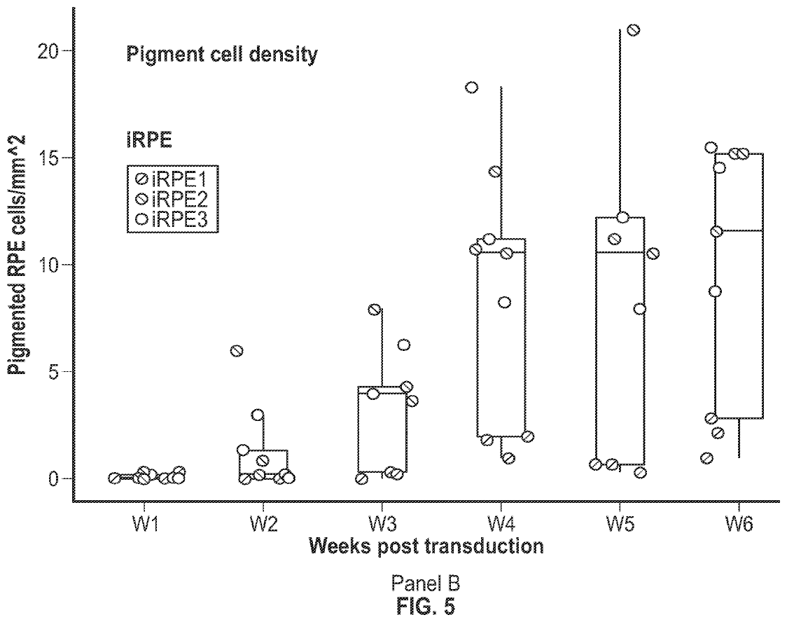

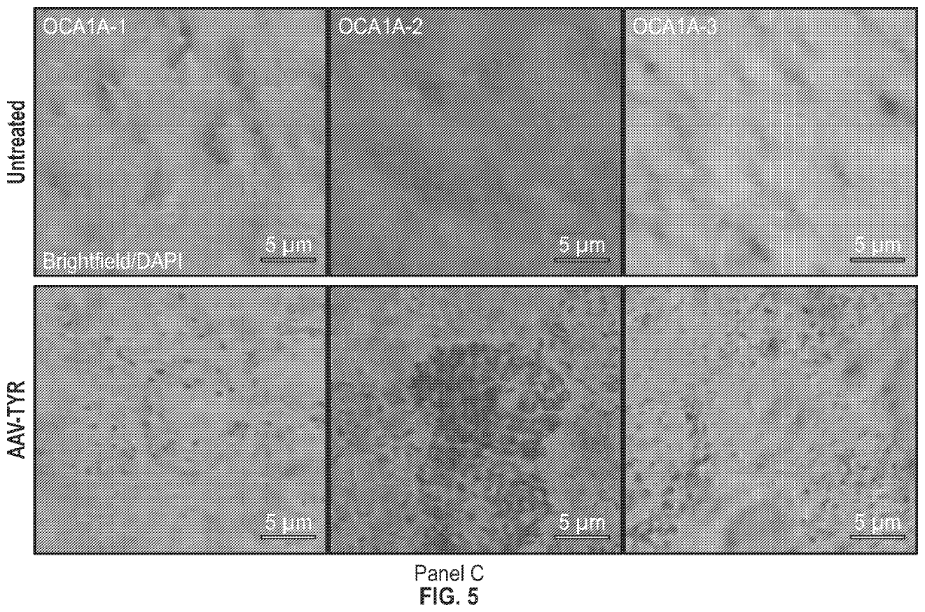

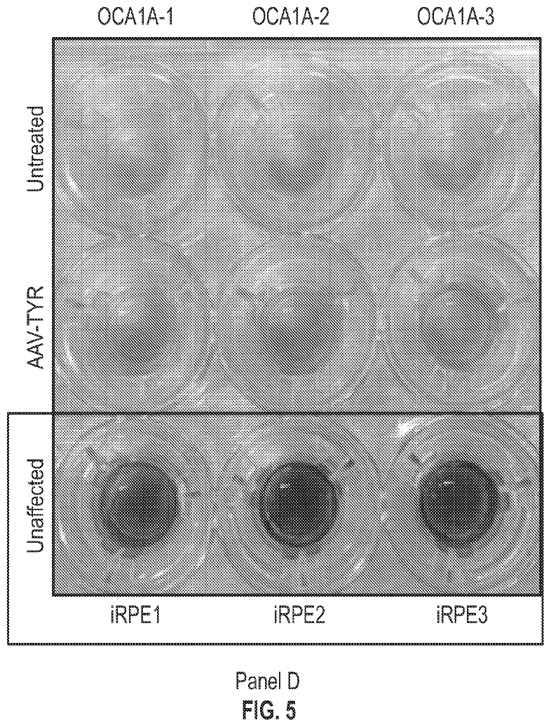

As noted, the derivation of iRPE from OCA1A patients recapitulates the pigmentation defects that are observed in OCA1A patients.⁷ AAV5 particles carrying the TYROSINASE gene under the control of hVMD2 (AAV5: hVMD2-TYR) were used to transduce OCA1A-iRPE monolayer tissue at the dose 10⁵ vector genomes/RPE cell (two unrelated OCA1A patients and one genetically-engineered TYR^{-/-} iRPE). RPE monolayer was washed 48 hrs post AAV transduction with 5% RPE media and cultured routinely for six weeks and imaged every week (FIG. 5, Panel A).

We observed pigmentation starting to accumulate as soon as 2 weeks post transduction, and an increase in pigmented cell density can be observed over time, in all the three OCA1A-iRPE as observed via brightfield imaging of live RPE monolayer tissue (FIG. 5, Panel B). We fixed the RPE monolayer tissue and performed high magnification transmitted light imaging using confocal microscopes and observed melanosomes in the form of dark black puncta in almost every cell, albeit at variable density per cell, in all three OCA1A-iRPE monolayer tissues (FIG. 5, Panel C). The overall pigment developed in the AAV-TYR treated OCA1A-iRPE monolayer tissue was observably lower as compared to culture-age matched iRPE monolayer tissue derived from unaffected individuals (FIG. 5, Panel D). We also observed differences in in response to AAV-TYR treatment among the three OCA1A-iRPE (FIG. 5, Panel B), where the degree of pigment accumulation and melanosome formation in OCA1A1-1 iRPE was reduced as compared to other two OCA1A iRPE lines. Similar differences were also observed with AAV-GFP treatment of pigmented iRPE lines (FIG. 1, Panels B and C).

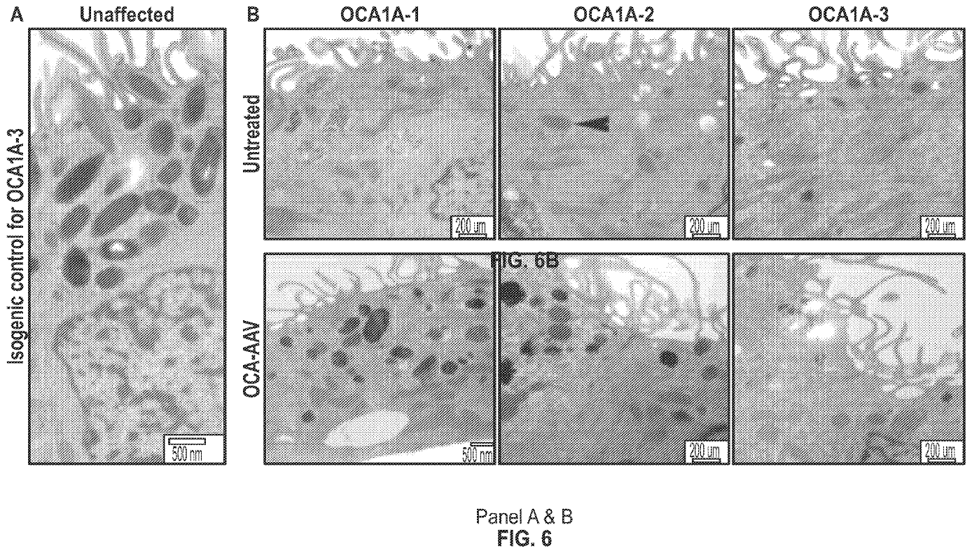

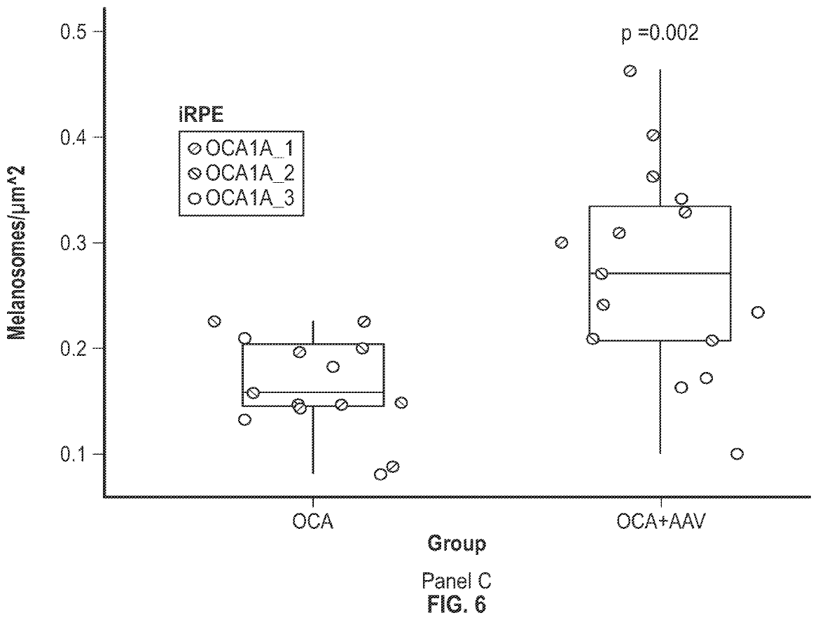

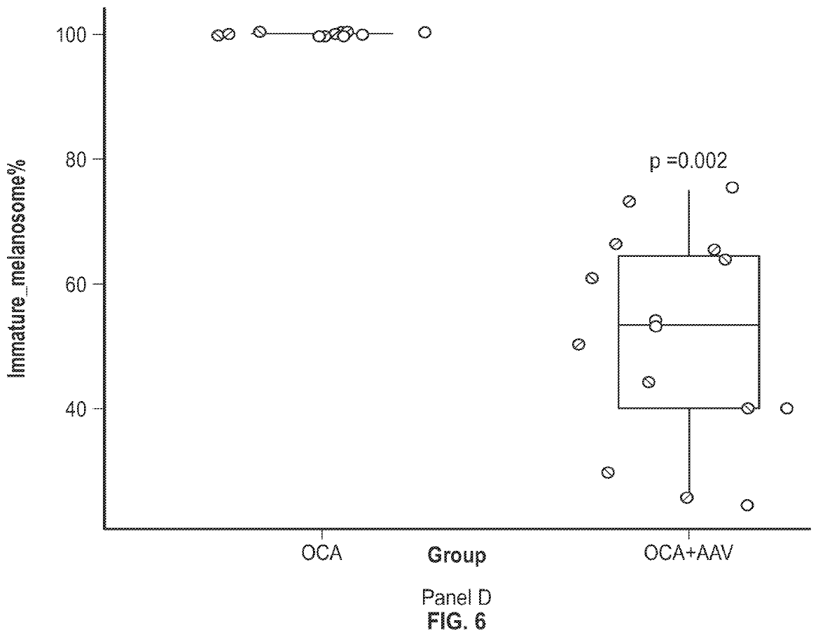

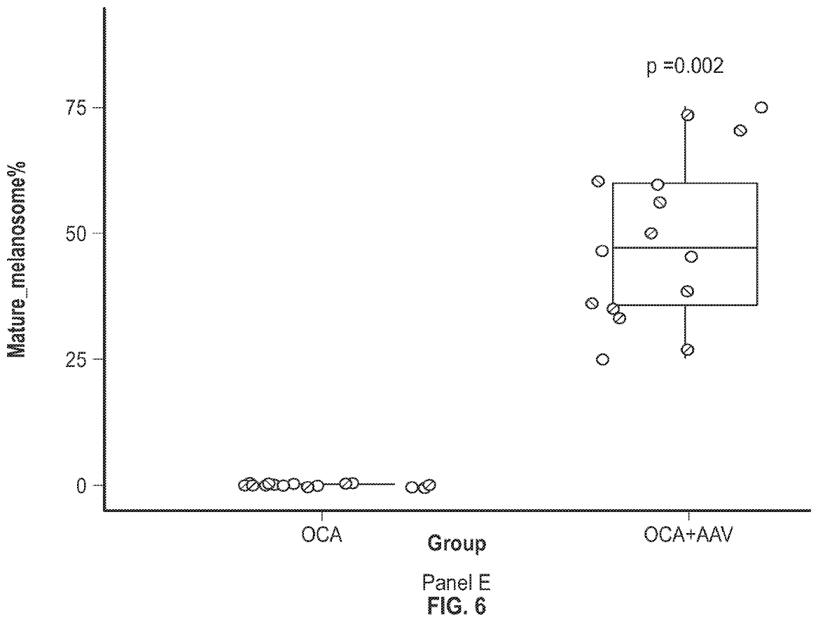

To further confirm that these black puncta are indeed melanosomes, we performed transmission electron microscopy (TEM) of untreated and AAV-TYR treated OCA1A-iRPE. As we have reported previously⁷ the unaffected iRPE cells exhibits mainly dark black spherical and oval shaped stage III and IV melanosomes and very few stage I and II pre melanosomes (FIG. 6, Panel A), whereas the OCA1A-iRPE are completely devoid of any mature melanosomes and contain only unpigmented pre-melanosomes (FIG. 6, Panel B, arrowhead).⁷ Similarly in our untreated OCA1A-iRPE we did not observe any pigmented mature melanosomes whereas in the AAV-TYR treated OCA1A-iRPE we observed mature melanosomes in all the three OCA1A-iRPE cells (FIG. 6, Panels B-E). We performed quantification of melanosomes in TEM micrographs and observed a significant increase in total melanosomes density (FIG. 6, Panel B, mature+pre-melanosome/μm²) in AAV-TYR treated OCA1A-iRPE compared to untreated iRPE. We also observed a significantly decreased percentage of pre-melanosomes and a concomitant increase of mature melanosomes in AAV-TYR treated OCA1A-iRPE cells as compared to untreated cells (FIG. 6, Panels D and E).

### Increased Mature Melanosome Marker Expression in AAV-TYR Treated OCA1A-iRPE

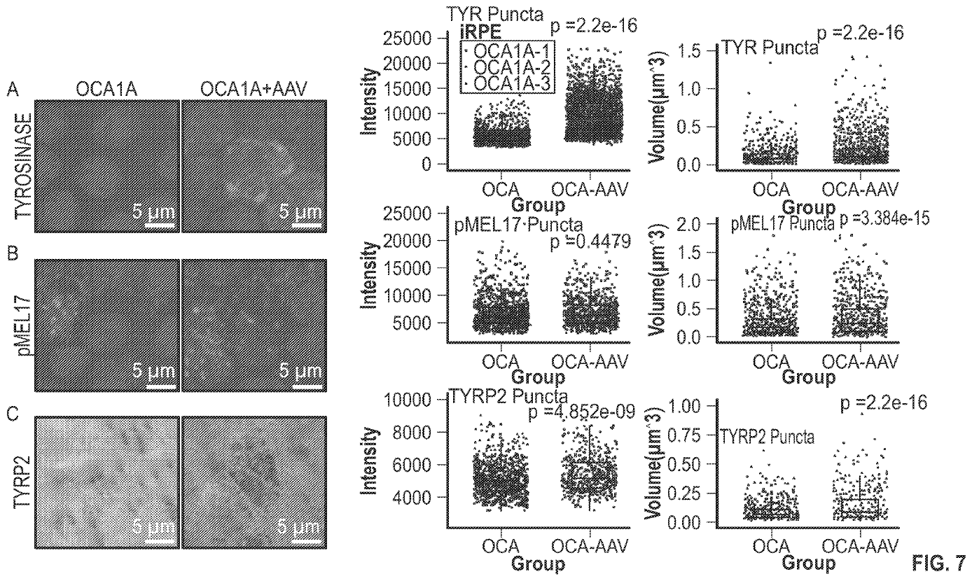

Post AAV-TYR transduction, OCA1A-iRPE monolayer were fixed and immuno-stained with antibody against melanosome proteins TYROSINASE and TYRP2, pMEL17 and imaged by confocal microscopy. We observed a significantly increased puncta fluorescence intensity and volume for TYROSINASE staining, in all the three AAV treated OCA1A-iRPE monolayers as compared to untreated OCA1A-iRPE suggesting production and accumulation of TYROSINASE (FIG. 7, Panel A). In our previous study,⁷ we reported that initial steps for melanogenesis (i.e. formation of pre-melanosomes) is not dependent on the presence of a functional TYROSINASE enzyme as we regularly observed pre-melanosomes in OCA1A-iRPE that lack functional TYROSINASE. Similarly, in the present study also we observed pre-melanosomes in untreated OCA1A-iRPE cells, as identified by pMEL17 immunostaining that specifically labels pre-melanosomes. AAV-TYR treated and untreated OCA1A-iRPE did not exhibit any changes to the puncta fluorescence intensity and volume (FIG. 7, Panel B). As observed using TEM, AAV-TYR induces pigment synthesis and accumulation in organelles resembling melanosomes. To confirm the identity of these pigmented organelles, we performed immuno-fluorescence staining with a marker of mature melanosomes, TYRP2. We observed a significant increase in the puncta fluorescence intensity and volume for TYRP2 staining, in all the three AAV treated OCA1A-iRPE monolayers as compared to untreated OCA1A-iRPE, suggesting increased formation and accumulation of mature melanosomes (FIG. 7, Panel C).

### EXAMPLE 3

This Example demonstrates the in vivo efficacy in inducing pigmentation, the specificity, and the toxicity of AAV5 particles carrying the human TYROSINASE gene under the RPE-specific human VMD2 gene promoter when administered to the eye via suprachoroidal injection.

### Suprachoroidal Delivery of TYROSINASE in Rat Eye Induces Pigmentation in RPE and Choroid

To study if we can induce pigmentation in vivo, we injected AAV particles carrying the hVMD2 promoter driven human TYROSINASE gene, in albino Sprague Dawley rat eyes via suprachoroidal injections. The rats were followed monthly by fundus examination to check for pigment accumulation.

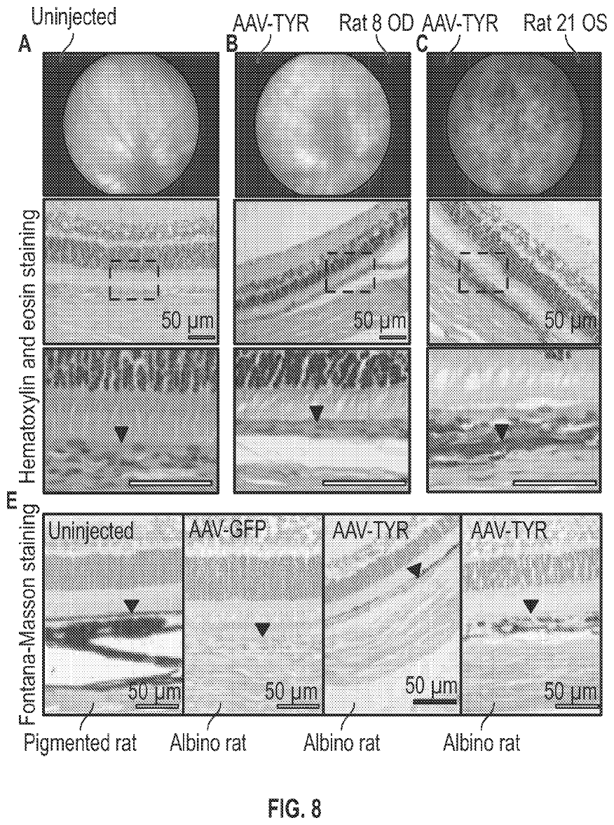

We did not observe any pigment accumulation in un-injected or AAV-GFP injected rat eyes as observed via visual examination, fundus imaging and histology (FIG. 8A). We tested two different doses of AAV-TYR (2×10⁸ vs 2×10¹⁰ VG) and then determined the pigment accumulation in RPE and choroid by measuring mean gray value of the fundus images. We did not observe any changes in the mean gray values at the two doses. In contrast, pigment accumulation was observed at least one-month post AAV-TYR transduction. Interestingly we could observe pigment accumulation not only in RPE where the VMD2 promoter is known to express, but also the choroid (FIG. 8, Panels B and C). When pigment accumulation occurred in RPE only it could not be detected with fundus imaging as it was too faint (FIG. 8, Panel B) whereas pigment accumulation in choroid was easily observable upon fundus imaging (FIG. 8, Panel C).

To further confirm that this pigment accumulation was occurring inside melanosomes, we performed TEM imaging and observed heavily pigmented melanosomes in the RPE of albino rat injected with AAV-TYR whereas pigmented melanosomes were never observed in albino rat RPE (N=2 eyes). As observed in OCA1A-iRPE we also observed non pigmented pre-melanosomes in albino rat RPE.

To further confirm that this pigment accumulation was due to melanin synthesis and accumulation, we performed Fontana-Masson staining (FIG. 8E). Un-injected pigmented rat eye sections exhibited characteristic black staining in the RPE and choroid region. AAV-GFP injected albino rat eyes were used as negative control and did not exhibit any staining. AAV-TYR injected rat eyes exhibited staining in the RPE only or in both RPE and choroid, thus suggesting that the AAV-TYR transduction led to melanin pigment accumulation in albino rat RPE and choroid.

### REFERENCES

All references, including publications, patent applications, and patents, cited herein are hereby incorporated by reference to the same extent as if each reference were individually and specifically indicated to be incorporated by reference and were set forth in its entirety herein. Also, while specifically not admitting to any of the following as constituting prior art, certain of the following documents are referenced herein above using the associated number in the following bibliographic list:
1. King, R. A., Hearing, V. J., Creel, D. J., & Oetting, W. S. (2019). Albinism. In D. L. Valle, S. Antonarakis, A. Ballabio, A. L. Beaudet, & G. A. Mitchell (Eds.), The Online Metabolic and Molecular Bases of Inherited Disease (Vol. 1-Book, Section). McGraw-Hill Education. ommbid. mhmedical. com/content. aspx?aid=1181477959
2. King, R., & Hearing, V. J. (2006). Oculocutaneous albinism. In The Pigmentary System:

Physiology and Pathophysiology. (p. 694). Blackwell Publishing.
3. Mabula, J. B., Chalya, P. L., Mchembe, M. D., Jaka, H., Giiti, G., Rambau, P., Masalu, N., Kamugisha, E., Robert, S., & Gilyoma, J. M. (2012). Skin cancers among Albinos at a University teaching hospital in Northwestern Tanzania: A retrospective review of 64 cases.

BMC Dermatology, 12(1), 5. doi./10.1186/1471-5945-12-5
4. Oculocutaneous Albinism—National Organization for Rare Disorders. (2015, August 18). NORD (National Organization for Rare Disorders). https://rarediseases.org/rare-diseases/oculocutaneous-albinism/
5. Cox, G., & Fulton, A. (2010). Albinism. In Ocular Disease (pp. 461-471).
6. Gargiulo, A., Bonetti, C., Montefusco, S., Neglia, S., Di Vicino, U., Marrocco, E., Corte, M. D., Domenici, L., Auricchio, A., & Surace, E. M. (2009). AAV-mediated Tyrosinase Gene Transfer Restores Melanogenesis and Retinal Function in a Model of Oculo-cutaneous Albinism Type I (OCA1). Molecular Therapy: The Journal of the American Society of Gene Therapy, 17(8), 1347-1354. doi./10.1038/mt.2009.112
7. George, A., Sharma, R., Pfister, T., Abu-Asab, M., Hotaling, N., Bose, D., DeYoung, C., Chang, J., Adams, D. R., Cogliati, T., Bharti, K., & Brooks, B. P. (2022). In vitro disease modeling of oculocutaneous albinism type 1 and 2 using human induced pluripotent stem cell-derived retinal pigment epithelium. Stem Cell Reports, 17(1), 173-186. doi./10.1016/j.stemcr.2021.11.016
8. Summers, C. G. (1996). Vision in albinism. Transactions of the American Ophthalmological Society, 94, 1095-1155.
9. Sparrow, J. R., Hicks, D., & Hamel, C. P. (2010). The retinal pigment epithelium in health and disease. Current Molecular Medicine, 10(9), 802-823. doi./10.2174/156652410793937813
10. Sharma, R., Bose, D., Maminishkis, A., & Bharti, K. (2020). Retinal Pigment Epithelium Replacement Therapy for Age-Related Macular Degeneration: Are We There Yet? Annual Review of Pharmacology and Toxicology, 60, 553-572. doi./10.1146/annurev-pharmtox-010919-023245
11. Russell, S., Bennett, J., Wellman, J. A., Chung, D. C., Yu, Z.-F., Tillman, A., Wittes, J., Pappas, J., Elci, O., McCague, S., Cross, D., Marshall, K. A., Walshire, J., Kehoe, T. L., Reichert, H., Davis, M., Raffini, L., George, L. A., Hudson, F. P., . . . Maguire, A. M. (2017). Efficacy and safety of voretigene neparvovec (AAV2-hRPE65v2) in patients with RPE65-mediated inherited retinal dystrophy: A randomised, controlled, open-label, phase 3 trial. Lancet (London, England), 390(10097), 849-860. doi./10.1016/S0140-6736(17)31868-8
12. Sharma, R., Bose, D., Montford, J., Ortolan, D., & Bharti, K. (2022). Triphasic developmentally guided protocol to generate retinal pigment epithelium from induced pluripotent stem cells. STAR Protocols, 3(3), 101582. doi. /10.1016/j.xpro.2022.101582
13. Yu, W., & Wu, Z. (2019). Use of AAV Vectors for CRISPR-Mediated In Vivo Genome Editing in the Retina. Methods in Molecular Biology (Clifton, N.J.), 1950, 123-139. doi./10.1007/978-1-4939-9139-6_7
14. Bigos, M. (2007). Separation Index: An Easy-to-Use Metric for Evaluation of Different Configurations on the Same Flow Cytometer. Current Protocols in Cytometry, 40(1), 1.21.1-1.21.6. doi./10.1002/0471142956.cy0121s40
15. Blaszczyk, W. M., Arning, L., Hoffmann, K.-P., & Epplen, J. T. (2005). A Tyrosinase missense mutation causes albinism in the Wistar rat. Pigment Cell Research, 18(2), 144-145. doi./10.1111/j.1600-0749.2005.00227.x
16. Furukawa, T., Morrow, E. M., Li, T., Davis, F. C., & Cepko, C. L. (1999). Retinopathy and attenuated circadian entrainment in Crx-deficient mice. Nature Genetics, 23(4), 466-470. doi./10.1038/70591
17. Harvey, P. S., King, R. A., & Summers, C. G. (2006). Spectrum of Foveal Development in Albinism Detected with Optical Coherence Tomography. Journal of American Association for Pediatric Ophthalmology and Strabismus, 10(3), 237-242. doi./10.1016/j. jaapos.2006.01.008
18. Yuodelis, C., & Hendrickson, A. (1986). A qualitative and quantitative analysis of the human fovea during development. Vision Research, 26(6), 847-855. doi./10.1016/0042-6989(86)90143-4
19. Cereso, N., Pequignot, M. O., Robert, L., Becker, F., De Luca, V., Nabholz, N., Rigau, V., De Vos, J., Hamel, C. P., & Kalatzis, V. (2014). Proof of concept for AAV2/5-mediated gene therapy in iPSC-derived retinal pigment epithelium of a choroideremia patient. Molecular Therapy. Methods & Clinical Development, 1, 14011. doi./10.1038/mtm.2014.11
20. Brydon, E. M., Bronstein, R., Buskin, A., Lako, M., Pierce, E. A., & Fernandez-Godino, R. (2019). AAV-Mediated Gene Augmentation Therapy Restores Critical Functions in Mutant PRPF31+/−iPSC-Derived RPE Cells. Molecular Therapy. Methods & Clinical Development, 15, 392-402. doi./10.1016/j. omtm.2019.10.014
21. Jia, R., Meng, X., Chen, S., Zhang, F., Du, J., Liu, X., & Yang, L. (2023). AAV-mediated gene-replacement therapy restores viability of BCD patient iPSC derived RPE cells and vision of Cyp4v3 knockout mice. Human Molecular Genetics, 32(1), 122-138. doi./10.1093/hmg/ddac181
22. Sinha, D., Steyer, B., Shahi, P. K., Mueller, K. P., Valiauga, R., Edwards, K. L., Bacig, C., Steltzer, S. S., Srinivasan, S., Abdeen, A., Cory, E., Periyasamy, V., Siahpirani, A. F., Stone, E. M., Tucker, B. A., Roy, S., Pattnaik, B. R., Saha, K., & Gamm, D. M. (2020). Human iPSC Modeling Reveals Mutation-Specific Responses to Gene Therapy in a Genotypically Diverse Dominant Maculopathy. American Journal of Human Genetics, 107(2), 278-292. doi./10.1016/j.ajhg.2020.06.011
23. Huang, Z., Zhang, D., Chen, S.-C., Jennings, L., Carvalho, L. S., Fletcher, S., Chen, F. K., & McLenachan, S. (2021). Gene replacement therapy restores RCBTB1 expression and cilium length in patient-derived retinal pigment epithelium. Journal of Cellular and Molecular Medicine, 25(21), 10020-10027. doi./10.1111/jcmm.16911
24. Rodrigues, A., Slembrouck-Brec, A., Nanteau, C., Terray, A., Tymoshenko, Y., Zagar, Y., Reichman, S., Xi, Z., Sahel, J.-A., Fouquet, S., Orieux, G., Nandrot, E. F., Byrne, L. C., Audo, I., Roger, J. E., & Goureau, O. (2022). Modeling PRPF31 retinitis pigmentosa using retinal pigment epithelium and organoids combined with gene augmentation rescue. NPJ Regenerative Medicine, 7(1), 39. doi./10.1038/s41536-022-00235-6
25. Ding, K., Shen, J., Hafiz, Z., Hackett, S. F., Silva, R. L. e, Khan, M., Lorenc, V. E., Chen, D., Chadha, R., Zhang, M., Everen, S. V., Buss, N., Fiscella, M., Danos, O., & Campochiaro, P. A. (2019). AAV8-vectored suprachoroidal gene transfer produces widespread ocular transgene expression. The Journal of Clinical Investigation, 129(11), 4901-4911. doi./10.1172/JCI129085
26. Wasnik, V. B., & Thool, A. R. (2022). Ocular Gene Therapy: A Literature Review With Focus on Current Clinical Trials. Cureus, 14(9), e29533. doi./10.7759/cureus.29533
27. Kansara, V., Muya, L., Wan, C., & Ciulla, T. A. (2020). Suprachoroidal Delivery of Viral and Nonviral Gene Therapy for Retinal Diseases. Journal of Ocular Pharmacology and Therapeutics, 36(6), 384-392. doi./10.1089/jop.2019.0126
28. Reichel, F. F., Peters, T., Wilhelm, B., Biel, M., Ueffing, M., Wissinger, B., Bartz-Schmidt, K. U., Klein, R., Michalakis, S., Fischer, M. D., & RD-CURE Consortium. (2018). Humoral Immune Response After Intravitreal But Not After Subretinal AAV8 in Primates and Patients. Investigative Ophthalmology & Visual Science, 59(5), 1910-1915. doi./10.1167/iovs.17-22494
29. Shen, J., Kim, J., Tzeng, S. Y., Ding, K., Hafiz, Z., Long, D., Wang, J., Green, J. J., & Campochiaro, P. A. (2020). Suprachoroidal gene transfer with nonviral nanoparticles. Science Advances, 6(27), eaba1606. doi./10.1126/sciadv.aba1606
30. Yiu, G., Chung, S. H., Mollhoff, I. N., Nguyen, U. T., Thomasy, S. M., Yoo, J., Taraborelli, D., & Noronha, G. (2020). Suprachoroidal and Subretinal Injections of AAV Using Transscleral Microneedles for Retinal Gene Delivery in Nonhuman Primates. Molecular Therapy-Methods & Clinical Development, 16, 179-191. doi./10.1016/j.omtm.2020.01.002
31. Chung, S. H., Mollhoff, I. N., Mishra, A., Sin, T.-N., Ngo, T., Ciulla, T., Sieving, P., Thomasy, S. M., & Yiu, G. (2021). Host Immune Responses after Suprachoroidal Delivery of AAV8 in Nonhuman Primate Eyes. Human Gene Therapy, 32(13-14), 682-693. doi./10.1089/hum.2020.281
32. XIPERE. (n.d.). XIPERE® (triamcinolone acetonide injectable suspension). XIPERE. Retrieved Feb. 9, 2023, from https://www.xipere.com/hcp/
33. Fung, S., & Syed, Y. Y. (2022). Suprachoroidal Space Triamcinolone Acetonide: A Review in Uveitic Macular Edema. Drugs, 82(13), 1403-1410. doi./10.1007/s40265-022-01763-7
34. Henry, C. R., Shah, M., Barakat, M. R., Dayani, P., Wang, R. C., Khurana, R. N., Rifkin, L., Yeh, S., Hall, C., & Ciulla, T. (2022). Suprachoroidal CLS-TA for non-infectious uveitis: An open-label, safety trial (AZALEA). The British Journal of Ophthalmology, 106(6), 802-806. doi./10.1136/bjophthalmol-2020-318019
35. Hanif, J., Iqbal, K., Perveen, F., Arif, A., Iqbal, R. N., Jameel, F., Hanif, K., Seemab, A., Khan, A. Y., & Ahmed, M. (2021). Safety and Efficacy of Suprachoroidal Injection of Triamcinolone in Treating Macular Edema Secondary to Noninfectious Uveitis. Cureus, 13(11), e20038.doi./10.7759/cureus.20038
36. Yeh, S., Kurup, S. K., Wang, R. C., Foster, C. S., Noronha, G., Nguyen, Q. D., Do, D. V., & Team, for the D. S. (2019). Suprachoroidal injection of triamcinolone acetonide, cls-ta, for macular edema due to noninfectious uveitis: A Randomized, Phase 2 Study (DOGWOOD). RETINA, 39(10), 1880. doi./10.1097/IAE.0000000000002279
37. Goldstein, D. A., Do, D., Noronha, G., Kissner, J. M., Srivastava, S. K., & Nguyen, Q. D. (2016). Suprachoroidal Corticosteroid Administration: A Novel Route for Local Treatment of Noninfectious Uveitis. Translational Vision Science & Technology, 5(6), 14. doi./10.1167/tvst.5.6.14
38. Khurana, R. N., Merrill, P., Yeh, S., Suhler, E., Barakat, M. R., Uchiyama, E., Henry, C. R., Shah, M., Wang, R. C., Kapik, B., & Ciulla, T. (2022). Extension study of the safety and efficacy of CLS-TA for treatment of macular oedema associated with non-infectious uveitis (MAGNOLIA). The British Journal of Ophthalmology, 106(8), 1139-1144. doi./10.1136/bjophthalmol-2020-317560
39. Yeh, S., Khurana, R. N., Shah, M., Henry, C. R., Wang, R. C., Kissner, J. M., Ciulla, T. A., Noronha, G., & PEACHTREE Study Investigators. (2020). Efficacy and Safety of Suprachoroidal CLS-TA for Macular Edema Secondary to Noninfectious Uveitis: Phase 3 Randomized Trial. Ophthalmology, 127(7), 948-955. doi./10.1016/j.ophtha.2020.01.006
40. Marashi, A., & Zazo, A. (2022). Suprachoroidal injection of triamcinolone acetonide using a custom-made needle to treat diabetic macular edema post pars plana vitrectomy: A case series. The Journal of International Medical Research, 50(4), 3000605221089807. doi./10.1177/03000605221089807
41. Zakaria, Y. G., Salman, A. G., Said, A. M. A., & Abdelatif, M. K. (2022). Suprachoroidal versus Intravitreal Triamcinolone Acetonide for the Treatment of Diabetic Macular Edema. Clinical Ophthalmology (Auckland, N.Z.), 16, 733-746. doi./10.2147/OPTH.S351853
42. Abdelshafy Tabl, A., Tawfik Soliman, T., Anany Elsayed, M., & Abdelshafy Tabl, M. (2022). A Randomized Trial Comparing Suprachoroidal and Intravitreal Injection of Triamcinolone Acetonide in Refractory Diabetic Macular Edema due to Epiretinal Membrane. Journal of Ophthalmology, 2022, e7947710. doi./10.1155/2022/7947710
43. Barakat, M. R., Wykoff, C. C., Gonzalez, V., Hu, A., Marcus, D., Zavaleta, E., & Ciulla, T. A. (2021). Suprachoroidal CLS-TA plus Intravitreal Aflibercept for Diabetic Macular Edema: A Randomized, Double-Masked, Parallel-Design, Controlled Study. Ophthalmology. Retina, 5(1), 60-70. doi./10.1016/j.oret.2020.08.007
44. Nawar, A. E. (2022). Modified Microneedle for Suprachoroidal Injection of Triamcinolone Acetonide Combined with Intravitreal Injection of Ranibizumab in Branch Retinal Vein Occlusion Patients. Clinical Ophthalmology, 16, 1139-1151. doi./10.2147/OPTH.S361636
45. Campochiaro, P. A., Wykoff, C. C., Brown, D. M., Boyer, D. S., Barakat, M., Taraborelli, D., Noronha, G., & Tanzanite Study Group. (2018). Suprachoroidal Triamcinolone Acetonide for Retinal Vein Occlusion: Results of the Tanzanite Study. Ophthalmology. Retina, 2(4), 320-328. doi./10.1016/j.oret.2017.07.013
46. Kohli, G. M., Shenoy, P., Halim, D., Nigam, S., Shetty, S., Talwar, D., & Sen, A. (2022). Safety and efficacy of suprachoroidal triamcinolone acetonide for the management of serous choroidal detachment prior to rhegmatogenous retinal detachment surgery: A Pilot study. Indian Journal of Ophthalmology, 70(4), 1302-1306. doi./10.4103/ijo.IJO_1788_21
47. Tabl, A. A., Elsayed, M. A., & Tabl, M. A. (2022). Suprachoroidal triamcinolone acetonide injection: A novel therapy for serous retinal detachment due to Vogt-Koyanagi Harada disease. European Journal of Ophthalmology, 32(6), 3482-3488. doi./10.1177/11206721221085420
48. Marashi, A., Baba, M., & Zazo, A. (2021). Managing solar retinopathy with suprachoroidal triamcinolone acetonide injection in a young girl: A case report. Journal of Medical Case Reports, 15(1), 577. doi./10.1186/s13256-021-03162-0
49. Jung, J. H., Kim, S. S., Chung, H., Hejri, A., & Prausnitz, M. R. (2022). Six-month sustained delivery of anti-VEGF from in-situ forming hydrogel in the suprachoroidal space. Journal of Controlled Release: Official Journal of the Controlled Release Society, 352, 472-484. doi./10.1016/j.jconrel.2022.10.036
50. Sher, I., Goldberg, Z., Bubis, E., Barak, Y., & Rotenstreich, Y. (2021). Suprachoroidal delivery of bevacizumab in rabbit in vivo eyes: Rapid distribution throughout the posterior segment. European Journal of Pharmaceutics and Biopharmaceutics, 169, 200-210. doi./10.1016/j.ejpb.2021.10.003
51. Kansara, V. S., Muya, L. W., & Ciulla, T. A. (2021). Evaluation of Long-Lasting Potential of Suprachoroidal Axitinib Suspension Via Ocular and Systemic Disposition in Rabbits. Translational Vision Science & Technology, 10(7), 19. doi./10.1167/tvst.10.7.19

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A genetic vector for expressing a gene of interest in tissues of the eye of a human patient, the vector comprising the gene of interest operably linked to a promoter suitable for effecting the expression of the gene of interest within the eye tissue of the human patient, (a) wherein the promoter comprises the human BESTROPHIN (hVMD2) gene promoter or a sequence having at least 95% identity to native hVMD2, or (b) wherein the gene of interest encodes the human TYROSINASE protein or a protein having at least 95% identity to native human TYROSINASE protein, or (c) both (a) and (b)

2. The vector of claim 1, which is derived from adeno-associated virus (AAV), optionally serotype AAV5.

3. The vector of claim 1, wherein the eye tissue comprises the retinal pigment epithelium (RPE).

4. The vector of claim 1, wherein the promoter comprises the human BESTROPHIN (hVMD2) gene promoter or a sequence having at least 95% identity to native hVMD 2.

5. The vector of claim 4, wherein the promoter comprises the human BESTROPHIN (hVMD2) gene promoter.

6. The vector of claim 1, wherein the gene of interest encodes the human TYROSINASE protein or a protein having at least 95% identity to native human TYROSINASE protein.

7. The vector of claim 6, wherein the gene of interest encodes the human TYROSINASE protein.

8. A composition comprising the vector of claim 1 and a pharmaceutically-acceptable excipient.

9. The composition of claim 8, which is formulated for injection.

10. A method of delivering a gene of interest to eye tissue within a human patient comprising administering the composition of claim 8 to the patient at a location and under conditions suitable to transduce cells within the eye tissue, whereby the gene of interest is expressed within the cells within the eye tissue.

11. (canceled)

12. The method of claim 10, wherein the eye tissue comprises the retinal pigment epithelium (RPE).

13. The method of claim 10, wherein the promoter comprises the human BESTROPHIN (hVMD2) gene promoter or a sequence having at least 95% identity to native hVMD 2.

14. The method of claim 13, wherein the promoter comprises the human BESTROPHIN (hVMD2) gene promoter.

15. The method of claim 10, wherein the gene of interest encodes the human TYROSINASE protein or a protein having at least 95% identity to native human TYROSINASE protein.

16. The method of claim 15, wherein the gene of interest encodes the human TYROSINASE protein.

17. The method of claim 10, wherein the composition is delivered to the patient by injection.

18. The method of claim 17, wherein the injection is via suprachoroidal injection.

19. The method of claim 10, wherein the promoter comprises the hVMD2 promoter, the gene of interest encodes the human TYROSINASE protein, the eye tissue comprises the RPE, the composition is delivered to the patient by suprachoroidal injection, and wherein the patient suffers from a clinically-defined pigmentation-related condition due to a loss-of-function mutation in the gene encoding the TYROSINASE protein, whereby the method or use comprises treating the patient, wherein infection of the cells within the RPE within the patient effects expression of the gene encoding the human TYROSINASE protein, whereby production of the protein within the RPE therapeutically treats the patient's pigmentation-related condition.

20. The method of claim 19, wherein the patient's pigmentation-related condition is oculocutaneous albinism type 1A (OCA1A).

21. The method of claim 10, wherein the patient is a human child undergoing foveal development.
